(19) United States
(12) Patent Application Publication
(10) Pub. No.: US 2022/0396831 A1
(43) Pub. Date: Dec. 15, 2022

(54) SYSTEMS AND METHODS FOR ASSESSING A TARGET MOLECULE

(71) Applicant: AXBIO INC., Santa Clara, CA (US)

(72) Inventors: Igor Constantin IVANOV, Danville, CA (US); Hui TIAN, Cupertino, CA (US)

(21) Appl. No.: 17/587,643

(22) Filed: Jan. 28, 2022

Related U.S. Application Data
(63) Continuation of application No. PCT/US20/44089, filed on Jul. 29, 2020.
(60) Provisional application No. 62/881,254, filed on Jul. 31, 2019.

(51) Int. Cl. C12Q 1/6869

(52) U.S. Cl. CPC C12Q 1/6869

(57) ABSTRACT

The present disclosure provides systems and methods for analyzing or identifying a target molecule. For example, a system of the present disclosure may comprise (i) a sensing electrode, (ii) a binding unit coupled to the sensing electrode and configured to bind at least a portion of the target molecule, and (iii) a dielectric material coupled to the sensing electrode and covering at least a portion of a surface of the sensing electrode. The sensor may be configured to detect one or more signals indicative of an impedance or impedance change in the sensor when the at least the portion of the target molecule is bound by the binding unit. The one or more signals may be usable to analyze or identify the target molecule.

### CROSS-REFERENCE

This application is a continuation of International Patent Application No. PCT/US20/44089, filed Jul. 29, 2020, which claims the benefit of U.S. Provisional Patent Application No. 62/881,254, filed Jul. 31, 2019, which is entirely incorporated herein by reference.

### BACKGROUND

Nucleic acid sequencing may be used to provide sequence information for a nucleic acid sample. Such sequence information may be helpful in diagnosing or treating a subject (e.g., an individual, a patient, etc.) of a condition (e.g., a disease). For example, nucleic acid sequence information of a subject may be used to identify, diagnose, or develop a treatment for one or more genetic diseases. In another example, nucleic acid sequence information of one or more pathogens may lead to treatment for one or more contagious diseases.

Detection of one or more rare sequence variants (e.g., mutations) may be valuable for healthcare. Detection of rare sequence variants may be important for and early detection of one or more pathological mutations. Detection of one or more cancer-associated mutations (e.g., point mutations) in clinical samples may improve identification of one or more minimal residual diseases during chemotherapy or detection of tumor cells in relapsing patients. Additionally, such detection of the mutation(s) may be important for assessment of exposure to environmental mutagens, monitoring endogenous DNA repair, or studying accumulation of one or more somatic mutations in aging individuals. The detection or rare sequence variant(s) may enhance prenatal diagnosis and enable characterization of fetal cells present in maternal blood.

Alternatively or in addition to, detection of small molecules and/or polypeptides (e.g., growth factors, enzymes, etc.) from a biological sample (e.g., blood, urine, tissue biopsies, etc.) may be used for identification or monitoring of one or more health conditions, such as cancer.

### SUMMARY

An aspect of the present disclosure provides a system for analyzing or identifying a target molecule, the system comprising: a sensor comprising (i) a sensing electrode, (ii) a binding unit coupled to the sensing electrode and configured to bind at least a portion of the target molecule, and (iii) a dielectric material coupled to the sensing electrode and covering at least a portion of a surface of the sensing electrode, wherein the sensor is configured to detect one or more signals indicative of an impedance or impedance change in the sensor when the at least the portion of the target molecule is bound by the binding unit, wherein the one or more signals are usable to analyze or identify the target molecule.

In some embodiments, the one or more signals are indicative of one or more members comprising: (i) electrical resistance or a change thereof in the sensor, (ii) electrical capacitance or a change thereof in the sensor, and (iii) an electrical inductance or a change thereof in the sensor.

In some embodiments of any one of the subject systems, the one or more signals are current or voltage. In some embodiments of any one of the subject systems, the one or more signals are not tunneling current.

In some embodiments of any one of the subject systems, an average cross-sectional dimension of the sensing electrode is no more than 20-fold greater than an average size of the target molecule. In some embodiments, the average cross-sectional dimension of the sensing electrode is no more than 2-fold greater than the average size of the target molecule.

In some embodiments of any one of the subject systems, an average cross-sectional dimension of the sensing electrode is smaller than the average size of the target molecule.

In some embodiments of any one of the subject systems, the binding unit is coupled to the sensing electrode via a conducting material.

In some embodiments of any one of the subject systems, the dielectric material is a self-assembled monolayer.

In some embodiments of any one of the subject systems, the target molecule comprises a tag, and the tag is configured to induce a change in the one or more signals.

In some embodiments of any one of the subject systems, the sensor further comprises a reference electrode in electrical communication with the sensing electrode, wherein the one or more signals are indicative of the impedance or impedance change between the sensing electrode and the reference electrode. In some embodiments, the sensing electrode and the reference electrode are configured to provides a first electric field along a first direction, and the system further comprises an additional electric field generator configured to apply a second electric field along a second direction that is different than the first direction. In some embodiments, the second direction is substantially orthogonal to the first direction.

In some embodiments of any one of the subject systems, the sensor is further configured to determine a residence time of the at least the portion of the target molecule on the binding unit.

In some embodiments of any one of the subject systems, the binding unit comprises one or more members selected from the group consisting of a small molecule, an enzyme, an antibody, a functional fragment thereof, and a functional variant thereof.

In some embodiments of any one of the subject systems, the target molecule comprises one or more members selected from the group consisting of a small molecule, a nucleotide, a polynucleotide, an amino acid, a peptide, a polypeptide, and a variant thereof.

Another aspect of the present disclosure provides a kit for analyzing or identifying a target molecule, the kit comprising: (i) any one of the subject systems comprising the sensor and (ii) instructions for providing a sample to be analyzed by the sensor, wherein the sample comprises or is suspected of having the target molecule.

A different aspect of the present disclosure provides a method for analyzing or identifying a target molecule, the method comprising: (a) providing a sensor comprising (i) a sensing electrode, (ii) a binding unit coupled to the sensing electrode and configured to bind at least a portion of the target molecule, and (iii) a dielectric material coupled to the sensing electrode and covering at least a portion of a surface of the sensing electrode; (b) detecting one or more signals indicative of an impedance or impedance change in the sensor when the at least the portion of the target molecule is bound by the binding unit; and (c) using the one or more signals to analyze or identify the target molecule.

In some embodiments, the one or more signals are indicative of one or more members comprising: (i) electrical resistance or a change thereof in the sensor, (ii) electrical capacitance or a change thereof in the sensor, and (ii) an electrical inductance or a change thereof in the sensor.

In some embodiments of any one of the subject methods, the one or more signals are current or voltage. In some embodiments of any one of the subject methods, the one or more signals are not tunneling current.

In some embodiments of any one of the subject methods, an average cross-sectional dimension of the sensing electrode is no more than 20-fold greater than an average size of the target molecule. In some embodiments, the average cross-sectional dimension of the sensing electrode is no more than 2-fold greater than the average size of the target molecule.

In some embodiments of any one of the subject methods, an average cross-sectional dimension of the sensing electrode is smaller than the average size of the target molecule.

In some embodiments of any one of the subject methods, the binding unit is coupled to the sensing electrode via a conducting material.

In some embodiments of any one of the subject methods, the dielectric material is a self-assembled monolayer.

In some embodiments of any one of the subject methods, the target molecule comprises a tag, wherein the tag is configured to induce a change in the one or more signals.

In some embodiments of any one of the subject methods, the sensor further comprises a reference electrode in electrical communication with the sensing electrode, and the one or more signals are indicative of the impedance or impedance change between the sensing electrode and the reference electrode. In some embodiments, the sensing electrode and the reference electrode provides a first electric field along a first direction, and the method further comprises using an additional electric field generator to apply a second electric field along a second direction that is different than the first direction. In some embodiments, the second direction is substantially orthogonal to the first direction.

In some embodiments of any one of the subject methods, the method further comprises determining a residence time of the at least the portion of the target molecule on the binding unit.

In some embodiments of any one of the subject methods, the binding unit comprises one or more members selected from the group consisting of a small molecule, an enzyme, an antibody, a functional fragment thereof, and a functional variant thereof.

In some embodiments of any one of the subject methods, the target molecule comprises one or more members selected from the group consisting of a small molecule, a nucleotide, a polynucleotide, an amino acid, a peptide, a polypeptide, and a variant thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A through 1E schematically illustrate an example of a sensor for analyzing or identifying a target molecule;

FIGS. 2A through 2E schematically illustrate another example of a sensor for analyzing or identifying a target molecule;

FIG. 3 schematically illustrates an example of a sensor with varied channel height;

FIGS. 4A and 4B schematically illustrate an example of a sensor with an additional electric field generator;

FIGS. 5A and 5B schematically illustrate an example of a sensor configured to detect redox potential of a target molecule;

FIG. 6 shows a computer system that is programmed or otherwise configured to implement methods provided herein; and

FIG. 7 shows an example process of analyzing or identifying a target molecule.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” can include plural references unless the context clearly dictates otherwise. For example, the term “a transmembrane receptor” can include a plurality of transmembrane receptors.

The term “about” or “approximately,” as used herein, can refer to within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “dielectric material,” as used herein, refers to an electrical insulating material that may be polarized by the action of an applied electric field. When a dielectric material is placed in electric field, electric charges may not flow through the dielectric material. In some cases, in such electric field, the dielectric material may exhibit dielectric polarization, in which positive charges may be displaced along the electric field and negative charges may shift in the opposite direction, thereby creating an internal electric field that may partly compensate the external electric field inside the dielectric material. Example of the dielectric material may include, but are not limited to, polyester, polyethylene, polypropylene, cloth (such as nylon), paper, laminate, glass, self-assembled monolayer (SAM), etc.

The terms “self-assembled monolayer” and “SAM,” as used interchangeably herein, refer to an ordered or a relatively ordered assembly (e.g., one or more layers) of molecules adsorbed on a surface (e.g., a surface of an electrode). A plurality of molecules within the SAM may be oriented approximately parallel to each other. A plurality of molecules within the SAM may be approximately perpendicular to the surface. Each of the molecules may include a functional group configured to adhere to the surface, and a portion configured to interact with one or more neighboring molecules (e.g., via hydrophobic interactions) in the SAM to form the relatively ordered array. The functional group may be configured to bind, covalently or non-covalently, to the surface. In an example, the functional group may be a thiol that covalently couples to a metal surface, such as a gold surface. In some cases, the SAM may be comprised of a single population of molecules or a mixed population of molecules. The mixed population of molecules may present (expose) a plurality of additional functionalities (e.g., biological functionalities, binding moieties, etc.). In some embodiments, the SAM can be used in a sensor (e.g., a biosensor, a chemical sensor, etc.) to detect molecules (e.g., small molecules, biological molecules, etc.) in a sample, such as a biological sample. Examples of the SAM may include, but are not limited to, 3-mercaptopropyltrimethoxysilane (3MPT), 3-aminopropltrimethoxysilane (APTES), p-aminophenyltrimethoxysilane (APTS), and 4-[2-(triethoxysilyl)ethyl]pyridine. In some cases, one or more conductive materials may be mixed within the SAM and in contact with the surface.

The term “conductive material,” as used herein, can refer to any material which can conduct electrical current, metals (e.g. tungsten, titanium, tantalum, aluminum, copper) and non-metals (e.g., conducting small molecules or conducting polymers). Examples of conducting polymers may include polyaniline, polypyrrole, polythiophene (e.g., poly(3,4-ethylenedioxythiophene)), polyfuran, polyphenylene (e.g., poly(p-phenylene vinylene)), functional variants thereof, and combinations thereof. In some cases, the degree of polymerization of a conducting polymer may be within a range of 10 to 100,000 monomer units.

The term “binding unit,” as used herein, generally refers to a molecule capable of interacting (e.g., binding) one or more target molecules. Such binding may be covalent and/or non-covalent (e.g., hydrogen bonding, hydrophobic interactions, etc.). The interaction between the binding unit and the target molecule(s) may be reversible or irreversible. Alternatively or in addition to, the binding unit may be configured to bind a tag (or probe) that is coupled to a target molecule. The target molecule may be a biomolecule. The target molecule may be a cell or one or more components or derivatives thereof of a cell.

The term “biomolecule,” as used herein, can refer to any molecule found in a biological system, a derivative thereof, or a functional variant thereof. The biomolecule may be naturally occurring or the result of an external disturbance of the system (e.g., a disease, poisoning, genetic manipulation, etc.), as well as synthetic analogs and derivatives thereof. Non-limiting examples of biomolecules may include amino acids (naturally occurring or synthetic), peptides, polypeptides, glycosylated and non-glycosylated proteins (e.g., polyclonal and monoclonal antibodies, receptors, interferons, enzymes, etc.), nucleosides, nucleotides, oligonucleotides (e.g., DNA, RNA, PNA oligos), polynucleotides (e.g., DNA, cDNA, RNA, etc.), carbohydrates, hormones, haptens, steroids, toxins, etc. Biomolecules may be isolated from natural sources, or they may be synthetic.

The term “cell,” as used herein, generally refers to a biological cell or cell derivative. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The terms “nucleotide,” “nucleobase,” and “base,” as used interchangeably herein, generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP), uridine triphosphate (UTP), and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein generally refers to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif., FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-1I1-dCTP, biotin-14-dCTP), and biotin-dUTP (e.g. biotin-11-dUTP, biotin-16-dUTP, biotin-20-dUTP).

Naturally-occurring nucleotides guanine, cytosine, adenine, thymine, and uracil may be abbreviated as G, C, A, T, and U, respectively. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or a variant thereof) or a pyrimidine (i.e., C, T or U, or a variant thereof). A subunit can enable individual nucleic acid bases or groups of bases (e.g., AA, TA, AT, GC, CG, CT, TC, GT, TG, AC, CA, or uracil-counterparts thereof) to be resolved.

The terms “polynucleotide,” “oligonucleotide,” “oligomer,” and “nucleic acid,” as used interchangeably herein, generally refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, complementary DNA (cDNA, such as double-strand cDNA (dd-cDNA) or single-stranded cDNA (ss-cDNA)), circulating tumor DNA (ctDNA), damaged DNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes (e.g., fluorescence in situ hybridization (FISH) probes), and primers. The sequence of nucleotides can be interrupted by non-nucleotide components. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide can be further modified after polymerization, such as by conjugation with a labeling component.

The term “gene,” as used herein, generally refers to a nucleic acid (e.g., DNA such as genomic DNA and cDNA) and its corresponding nucleotide sequence that is involved in encoding an RNA transcript. The term “gene” as used herein with reference to genomic DNA may include intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. The genes may not encode a polypeptide, for example, ribosomal RNA genes (rRNA) and transfer RNA (tRNA) genes. The term “gene” may not only include the transcribed sequences, but also non-transcribed regions including upstream and downstream regulatory regions, enhancers and promoters. A gene may be an “endogenous gene” or a native gene in its natural location in the genome of an organism. A gene may be an “exogenous gene” or a non-native gene. A non-native gene may be a gene not normally found in the host organism but which is introduced into the host organism by gene transfer (e.g., transgene). A non-native gene may be a naturally occurring nucleic acid or polypeptide sequence that comprises mutations, insertions and/or deletions (e.g., non-native sequence).

The term “mutation,” as used herein, generally refers to a change in the sequence of nucleotides of a normally conserved nucleic acid sequence resulting in the formation of a mutant as differentiated from the normal (unaltered) or wild type sequence. A position (e.g., relative to a gene or a sample polynucleotide) and sequence of the mutation may be undetermined prior to sequencing. Alternatively, a position (e.g., relative to a gene or a sample polynucleotide) and sequence of the mutation may be determined prior to sequencing, in which case the sequencing may be performed to detect a presence or absence of the mutation in the sample polynucleotide. A mutation can comprise a base-pair substitution (e.g. single nucleotide substitution) and a frame-shift mutation. The frame-shift mutation may require insertion or deletion of one to several nucleotide pairs.

The term “probe,” as used herein, generally refers to a nucleotide or polynucleotide that is tagged with a maker (e.g., a fluorescent marker) useful for detecting or identifying its corresponding target nucleotide or polynucleotide in a hybridization reaction by hybridization with a corresponding target sequence. The terms “nucleotide probe, “nucleotide tag,” and “tagged nucleotide,” as used interchangeable herein, generally refer to a probe having a single nucleotide. The terms “polynucleotide probe, “polynucleotide tag,” and “tagged polynucleotide,” as used interchangeable herein, generally refer to a probe having polynucleotide. A polynucleotide probe may be tagged with at least one marker (e.g., one marker per each nucleotide of the polynucleotide probe). A probe may be hybridizable to one or more target nucleotides or polynucleotides. A polynucleotide probe can be entirely complementary to one or more target polynucleotides in a sample, or contain one or more nucleotides that are not complementary (i.e., a mismatch) to one or more nucleotides of the one or more target polynucleotides in the sample.

In some embodiments, the maker may be a redox species. The term “redox species,” as used herein, generally refers to a molecule or compound or a portion thereof (e.g., a molecular or functional moiety of a molecular or compound) that can be oxidized and/or reduced (i.e., “redox”) during or upon electrical stimulation (e.g., during or upon application of an electrical potential), or can undergo a Faradaic reaction. In an example, a redox species may comprise one or more molecular moieties that accept and/or donate one or more electrons depending on its redox state. In some cases, the redox species may form part (e.g., a molecular moiety) of a small molecule, a compound, a polymer molecule, or can exist as an individual molecule or compound. Examples of the redox species may include imidazolium, pyrrolidinium, tetraalkylammonium, [OTf]-, [FAP]-, [PF6]-, [BF4]-, [DCA]-, [NTf2]-, [FSI]-, [B(CN)4]-, ferrocene (Fc), derivatives thereof, functional variants thereof, and combinations thereof. Examples of Fc derivatives may include, methyl ferrocene, dimethyl ferrocene, ethyl ferrocene, propyl ferrocene, n-butyl ferrocene, t-butyl ferrocene, and 1,1-dicarboxylate ferrocene.

Identical nucleotides may be tagged with a same marker. Alternatively, identical nucleotides may be tagged with different markers. For example, a first nucleotide A may be tagged with a first maker, and a second nucleotide A may be tagged with a second maker, wherein the first maker and the second maker are different. In cases where a sensor can detect and distinguish the first maker and the second maker apart from each other, using a plurality of makers for the same nucleotide may help resolve sequencing of identical nucleotides that are presented in a sequential manner.

The terms “complement,” “complements,” “complementary,” and “complementarity,” as used interchangeably herein, generally refer to a sequence that is fully complementary to and hybridizable to the given sequence. A sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. The respective lengths may comprise a region of at least 2, 3, 4, 5, 6, 7, 8, 9, 10, 11, 12, 13, 14, 15, 16, 17, 18, 19, 20, 21, 22, 23, 24, 25, 30, 35, 40, 45, 50, or more nucleotides.

Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods

The term “hybridization” as used herein, generally refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence may be referred to as the “complement” of the first sequence. The term “hybridizable,” as applied to a polynucleotide, generally refers to the ability of the polynucleotide to form a complex that is stabilized via hydrogen bonding between the bases of the nucleotide residues in a hybridization reaction.

The term “target polynucleotide,” as used herein, generally refers to a nucleic acid molecule or polynucleotide in a population of nucleic acid molecules having a target sequence. in which the presence, amount, and/or nucleotide sequence, or changes in one or more of these, are desired to be determined. The term “target sequence” generally refers to a nucleic acid sequence on a single strand of nucleic acid. The target sequence may be a portion of a gene, a regulatory sequence, genomic DNA, cDNA, ctDNA, RNA including mRNA, miRNA, rRNA, or others. The target sequence may be a target sequence from a sample or a secondary target such as a product of an amplification reaction. The target polynucleotide may be part of a gene (or a fragment thereof) that comprises one or more mutations.

The term “target site,” as used herein, generally refers to a polynucleotide sequence that comprises the target polynucleotide (or a target nucleotide). The target polynucleotide (or target nucleotide) of the target site may be one or more sequence variants. Examples of the one or more sequence variants may include a single nucleotide variation, insertion or deletion of one or more nucleotides (e.g., sequential or non-sequential nucleotides), copy-number variation (CNV) comprising one or more repeats of one or more nucleotides (e.g., a CNV with a mean size of at least 1, 5, 10, 50, 100, 150, 200, or more kilobases (kb); a CNV with a mean size of at most 200, 150, 100, 50, 10, 5, 1, or fewer kb), and microsatellite instability (MSI). The target site may comprise at least 1, 2, 3, 4, 5, 6, 7, 8, 9, 10, 15, 20, 25, 30, 40, 50, 100, 200, 300, 400, 500, 1,000, 5,000, 10,000, 50,000, 100,000, 500,000, or more nucleotides. The target site may comprise at most 500,000, 100,000, 50,000, 10,000, 5,000, 1,000, 500, 400, 300, 200, 100, 50, 40, 30, 25, 20, 15, 10, 9, 8, 7, 6, 5, 4, 3, 2, or 1 nucleotide. The target site may comprise at least 1, 2, 3, 4, 5, 6, 7, 8, 9, 10, 15, 20, 25, 30, 40, 50, 100, 200, 300, 400, 500, 1,000, 5,000, 10,000, 50,000, 100,000, 500,000, or more nucleotides than the target polynucleotide. The target site may comprise at most 500,000, 100,000, 50,000, 10,000, 5,000, 1,000, 500, 400, 300, 200, 100, 50, 40, 30, 25, 20, 15, 10, 9, 8, 7, 6, 5, 4, 3, 2, or 1 nucleotide than the target polynucleotide. In some examples, the target site may be the target polynucleotide.

The term “stringent condition,” as used herein, generally refers to one or more hybridization conditions under which a nucleic acid having complementarity to a target sequence predominantly hybridizes with a target sequence, and substantially does not hybridize to non-target sequences. Stringent conditions may be sequence-dependent, and may vary depending on a number of factors. In some cases, the longer the sequence, the higher the temperature at which the sequence may specifically hybridize to its target sequence.

The term “recognition moiety,” as used herein, generally refers to a molecule (e.g., a small molecule, a polynucleotide, a protein, a variation thereof, or a combination thereof) that is capable of interacting with a nucleic acid sequence, i.e., a “recognition sequence” or “a recognition site,” such as a desired (or target) nucleic acid sequence. The recognition moiety may comprise a domain (e.g., a component comprising the domain) capable of binding (e.g., hybridizing) to the recognition sequence. Such domain may comprise one or more amino acids, one or more nucleotides, a variation thereof, or a combination thereof. Alternatively or in addition to, the recognition moiety may associate with (e.g., bind to) a secondary molecule comprising such domain. In some examples, the recognition moiety may comprise a nucleic acid molecule capable of hybridizing to the recognition sequence. In some examples, the recognition moiety may comprise a component that exhibits a particular biological activity comprising, but are not limited to, one or more activities of a nuclease (e.g., double-stranded nuclease), nickase, transcriptional activator, transcriptional repressor, nucleic acid methylation enzyme, nucleic acid demethylation enzyme, and recombinase. The recognition sequence may comprise at least 3, 4, 5, 6, 7, 8, 9, 10, 11, 12, 13, 14, 15, 16, 17, 18, 19, 20, 21, 22, 23, 24, 25, 30, 35, 40, 45, 50, or more nucleotides. The recognition sequence may comprise at most 50, 45, 40, 35, 30, 25, 24, 23, 22, 21, 20, 19, 18, 17, 16, 15, 14, 13, 12, 11, 10, 9, 8, 7, 6, 5, 4, 3, or fewer nucleotides.

The recognition moiety may be used to isolate a desired molecule comprising the desired nucleic acid sequence from a plurality of molecules (e.g., a plurality of nucleic acid molecules). The recognition moiety may be used to enrich for a desired molecule comprising the desired nucleic in a composition or a reaction mixture. In some examples, the recognition moiety may be captured by a capturing system (e.g., magnetic beads) via one or more interactions (e.g., avidin-biotin binding, magnetic binding, etc.). In an example, the recognition moiety may comprise biotin, which may complex with a streptavidin magnetic bead for isolation or enrichment.

Examples of the recognition moiety can include CRISPR-associated (Cas) systems (e.g., Cas proteins, including catalytically active or inactive Cas polypeptides); zinc finger nucleases (ZFN); transcription activator-like effector nucleases (TALEN); meganucleases; RNA-binding proteins (RBP); Cas RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); a variant thereof; and a combination thereof. The recognition moiety may include a polynucleotide (e.g., at least 2, 3, 4, 5, 6, 7, 8, 9, 10, or more nucleotide-long sequence) that can be captured by a capturing system via one or more interactions, such as, for example, a polynucleotide sequence tagged with a biotin that can be captured by one or more avidin-functionalized magnetic beads. At least a portion of the polynucleotide may share complementarity to a recognition sequence of a target nucleic acid molecule.

The term “nickase,” as used herein, generally refers to a molecule (e.g., an enzyme) that cleaves one strand of a double-stranded nucleic acid molecule (i.e., “nicks” a double-stranded molecule). The nickase may be a nuclease that cleaves only a single DNA strand, either due to its natural function or because it has been engineered (e.g., modified by mutation and/or deletion of one or more nucleotides) to cleave only a single DNA strand. The nickase may be a nicking enzyme (e.g., a restriction endonuclease, nicking endonuclease, etc.). The nickase may bind to a nicking site of a double-stranded nucleic acid molecule to create a nick (or a gap) in one strand of the double-stranded nucleic acid molecule. The nick may be generated within the nicking site. Alternatively, the nick may be generated adjacent to the nicking site. In some cases, the nickase may bind to a nickase binding site that is adjacent to the nicking site. The nick may be the length of at least 1, 2, 3, 4, 5, 6, 7, 8, 9, 10, or more nucleotides. The nick may be the length of at most 10, 9, 8, 7, 6, 5, 4, 3, 2, or 1 nucleotide. Examples of the nickase can include Cas systems (e.g., a Cas nickase, such as Cas9n), N.Alw I, Nb.BbvCl, Nt.BbvCl, Nb.BsmI, Nt.BsmAI, Nt.BspQl, Nb.BsrDI, Nt.BstNBI, Nb.BstsCI, Nt.CviPII, Nb.Bpu 1 OI, Nt.Bpu 1 OI and Nt,Bst9I, variations thereof, and combinations thereof. In some examples, a nucleic acid molecule (e.g., a double-stranded nucleic acid molecule or a single-stranded nucleic acid molecule that is self-complementary) may comprise at least one nicking site that already includes at least one nick.

The terms “CRISPR-associated system,” “Cas system,” and “Cas complex,” as used interchangeably herein, generally refer to a two component ribonucleoprotein complex with guide RNA (gRNA) and a Cas polypeptide or protein (e.g., a Cas endonuclease, a catalytic or a non-catalytic derivative thereof, etc.), or other protein having endonuclease activity. The term “CRISPR” refers to the Clustered Regularly Interspaced Short Palindromic Repeats and the related system thereof. At least a portion of the gRNA can have complementarity to at least a portion of the target region. The target region can comprise a “protospacer” and a “protospacer adjacent motif” (PAM), and both domains may be needed for a nuclease activity (e.g., cleavage) of the Cas polypeptide. The protospacer may be referred to as a target site (or a genomic target site). The gRNA may pair with (or hybridize) the opposite strand of the protospacer (binding site) to direct the Cas polypeptide to the target region. The PAM site generally refers to a short sequence recognized by the Cas polypeptide and, in some cases, can be required for the nuclease (or nickase) activity. The sequence and number of nucleotides for the PAM site can differ depending on the type of the Cas enzyme.

The Cas polypeptide may comprise a nuclease (or nickase) activity, and the gRNA may interact with the Cas polypeptide to direct the nuclease (or nickase) activity of the Cas polypeptide to a desired target region. Alternatively, the Cas polypeptide may be non-catalytic and may not comprise a nuclease activity. The non-catalytic Cas polypeptide may be referred to as a dead or inactive Cas (dCas).

A Cas protein may comprise a protein of or derived from a CRISPR-associated type I, type II, or type III system, which may have an RNA-guided polynucleotide-binding or nuclease activity. Examples of suitable Cas proteins include Cas3, Cas4, Cas5, Cas5e (or CasD), Cas6, Cas6e, Cas6f, Cas7, Cas8a1, Cas8a2, Cas8b, Cas8c, Cas9 (i.e., Csn1 and Csx12), Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, homologues thereof, and modified versions (e.g., catalytic or non-catalytic) thereof. In some cases, a Cas protein may comprise a protein of or derived from a CRISPR-associated type V or type VI system, such as Cpf1 (or Cas12a), C2c1 (or Cas12b), C2c2, homologues thereof, and modified versions (e.g., catalytic or non-catalytic) thereof.

Although certain examples herein refer to a Cas protein, other proteins with endonuclease activity may be used. Such other proteins may not be Cas protein, but may be configured for use with a gRNA, for example.

The Cas polypeptide or protein may be engineered to modify the nuclease activity to a nickase activity. For example, an aspartate-to-alanine substitution (D10A) in the RuvC I catalytic domain of Cas9 from S. pyogenes can convert Cas9 from a nuclease that cleaves both strands to a Cas9n nickase that cleaves only a single strand. The Cas9n nickase mutants can introduce gRNA-targeted single-strand breaks in DNA instead of the double-strand breaks created by wild type Cas polypeptides. Other examples of mutations that render Cas9a nickase can include H840A, N854A, and N863A.

The term “guide RNA (gRNA),” as used herein, generally refers to an RNA molecule that can bind to a Cas polypeptide and aid in targeting the Cas polypeptide to a specific location within a target nucleic acid region (e.g., a DNA or a gene). A degree of complementarity between a gRNA and the specific location within the target nucleic acid region can be at least 50%, 60%, 75%, 80%, 85%, 90%, 95%, 97.5%, 99%, or more. A guide RNA can comprise a CRISPR RNA (crRNA) segment and a trans-activating crRNA (tracrRNA) segment. The terms “crRNA” and “crRNA segment,” as used interchangeably herein, generally refer to an RNA molecule or portion thereof that includes a polynucleotide-targeting guide sequence, a stem sequence, and, optionally, a 5′-overhang sequence. The terms “tracrRNA” and “tracrRNA segment,” as used interchangeably herein, generally refer to an RNA molecule or portion thereof that includes a protein-binding segment (e.g., the protein-binding segment capable of interacting with a CRISPR-associated protein, such as a Cas9). In some cases, the guide RNA may be a single guide RNA (sgRNA), where the crRNA segment and the tracrRNA segment are located in the same RNA molecule. The gRNA may comprise one or more peptide nucleic acids.

The crRNA may comprise at least 15, 16, 17, 18, 19, 20, 21, 22, 23, 24, 25, 30, 35, 40, or more RNA bases. The crRNA may comprise at most 40, 35, 30, 25, 24, 23, 22, 21, 20, 19, 18, 17, 16, 15, or fewer RNA bases. The target nucleic acid sequence of the gRNA of the Cas system may comprise at least 15, 16, 17, 18, 19, 20, 21, 22, 23, 24, 25, 30, 35, 40, or more DNA bases. The target nucleic acid sequence of the gRNA of the Cas system may comprise at most 40, 35, 30, 25, 24, 23, 22, 21, 20, 19, 18, 17, 16, 15, or fewer DNA bases. The crRNA sequence can be selected to target any target sequence. A target sequence can be a sequence within a genome of a cell. A target sequence can include those that are unique in the target genome.

The term “polymerase,” as used herein, generally refers to an enzyme (e.g., natural or synthetic) capable of catalyzing a polymerization reaction. Examples of polymerases can include a nucleic acid polymerase (e.g., a DNA polymerase or an RNA polyemrase), a transcriptase, and a liage. A polymerase can be a polymerization enzyme. The term “DNA polymerase” generally refers to an enzyme capable of catalyzing a polymerization reaction of DNA.

The term “linked polymerase,” as used herein, generally refers to a polymerase such as a DNA polymerase that is coupled to (e.g., fused to) a linker. The linker may be capable of coupling to (e.g., binding or conjugating to) another entity (e.g., a nanopore, such as a protein nanopore or a solid state nanopore).

The terms “sequence variant” and “sequencing variant,” as used interchangeably herein, generally refer to any variation in sequence relative to one or more reference sequences. Typically, a sequence variant occurs with a lower frequency than a reference sequence for a given population of individuals for whom the reference sequence is provided. For example, a particular bacterial genus may have a consensus reference sequence for the 16S rRNA gene, but individual species within that genus may have one or more sequence variants within the gene or a portion of a gene that are useful in identifying that species in a population of bacteria. As a further example, sequences for multiple individuals of the same species or multiple sequencing reads for the same individual may produce a consensus sequence when optimally aligned, and sequence variants with respect to that consensus may be used to identify mutants in the population indicative of dangerous contamination. In general, a “consensus sequence” refers to a nucleotide sequence that reflects the most common choice of base at each position in the sequence where the series of related nucleic acids has been subjected to intensive mathematical and/or sequence analysis, such as optimal sequence alignment according to any of a variety of sequence alignment algorithms. A reference sequence may be a single reference sequence, such as a predetermined genomic sequence of a single individual. A reference sequence can be a consensus sequence formed by aligning multiple sequences, such as predetermined genomic sequences of multiple individuals serving as a reference population, or multiple sequencing reads of polynucleotides from the same individual. A reference sequence can be a consensus sequence formed by optimally aligning the sequences from a sample under analysis, such that a sequence variant represents a variation relative to corresponding sequences in the same sample. A sequence variant can occur with a low frequency in the population (also referred to as a “rare” sequence variant). For example, a sequence variant may occur with a frequency of or less than 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. A sequence variant can occur with a frequency of or less than 0.1%.

A sequence variant can be any variation with respect to a reference sequence. A sequence variation may consist of a change in, insertion of, or deletion of a single nucleotide, or of a plurality of nucleotides such as, for example, 2, 3, 4, 5, 6, 7, 8, 9, 10, or more nucleotides. Where a sequence variant comprises two or more nucleotide differences, the nucleotides that are different may be contiguous with one another or discontinuous. Examples of types of sequence variants include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g., methylation differences).

The term “sequencing,” as used herein, generally refers to a procedure for determining the order in which nucleotides occur in a target nucleotide sequence. Methods of sequencing can comprise high-throughput sequencing, such as, for example, next-generation sequencing (NGS). Sequencing may be, whole-genome sequencing or targeted sequencing. Sequencing may be single molecule sequencing or massively parallel sequencing. Next-generation sequencing methods can be useful in obtaining millions of sequences in a single run. In an example, sequencing may be performed using one or more nanopore sequencing methods, e.g., sequencing-by-synthesis, sequencing-by-ligation, or sequencing-by-cleavage.

The term “nanopore,” as used herein, generally refers to a pore, channel, or passage formed or otherwise provided in a membrane. The membrane may be an organic membrane, such as a lipid bilayer, or a synthetic membrane, such as a membrane formed of a polymeric material such as a protein nanopore. The membrane may be a solid state membrane (e.g., silicon substrate). The nanopore may be disposed adjacent or in proximity to a sensing circuit or an electrode coupled to a sensing circuit, such as, for example, a complementary metal-oxide semiconductor (CMOS) or field effect transistor (FET) circuit. The nanopore may be part of the sensing circuit. A nanopore can have a characteristic width or diameter, for example, on the order of about 0.1 nanometer (nm) to 1000 nm. A nanopore can be a biological nanopore, solid state nanopore, hybrid biological-solid state nanopore, a variation thereof, or a combination thereof. Examples of the biological nanopore include, but are not limited to, OmpG from E. coli, sp., Salmonella sp., Shigella sp., and Pseudomonas sp., and alpha hemolysin (α-hemolysin) from S. aureus sp., MspA from M. smegmatis sp, a functional variant thereof, or a combination thereof. Sequencing may comprise forward sequencing and/or reverse sequencing. Examples of the solid state nanopore include, but are not limited to, silicon nitride, silicon oxide, graphene, molybdenum sulfide, a functional variant thereof, or a combination thereof. The solid state nanopore may be fabricated by high-energy beam manufacturing, imprinting (e.g., nanoimprinting), laser ablation, chemical etching, plasma etching (e.g., oxygen plasma etching), etc.

The term “nanopore sequencing complex,” as used herein, generally refers to a nanopore linked or coupled to an enzyme, e.g., a polymerase, which in turn is associated with a polymer, e.g., a polynucleotide template. The nanopore sequencing complex may be positioned in a membrane, e.g., a lipid bilayer, where it functions to identify polymer components, e.g., nucleotides or amino acids.

The terms “nanopore sequencing” and “nanopore-based sequencing,” as used interchangeably herein, generally refer to a method that determines the sequence of a polynucleotide with the aid of a nanopore. In some cases, the sequence of the polynucleotide may be determined in a template-dependent manner. In some cases, the methods, systems, or compositions disclosed herein may not be limited to any particular nanopore sequencing method, system, or device.

The term “barcode,” as used herein, generally refers to a predetermined nucleic acid sequence that allows some feature of a polynucleotide with which the barcode is associated to (e.g., a polynucleotide comprising at least a portion of the barcode or a polynucleotide having complementarity to at least a portion of the barcode) be identified. In some examples, the feature of the polynucleotide to be identified may be the sample from which the polynucleotide is derived. A barcode may be at least about 3, 4, 5, 6, 7, 8, 9, 10, 11, 12, 13, 14, 15, 16, 17, 18, 19, 20, or more nucleotides in length. A barcode may be at most 20, 19, 18, 17, 16, 15, 14, 13, 12, 11, 10, 9, 8, 7, 6, 5, 4, 3, or 2 nucleotides in length. A barcode associated with polynucleotides from a first sample may be different (e.g., different sequences and/or different lengths) than the barcode associated with polynucleotides from a second sample that is different than the first sample. In such a case, identification of the barcode in the respective polynucleotides may help identify the sample source of one or more of the polynucleotides. Thus, different samples with different barcodes can be analyzed (e.g., sequenced) together (e.g., in the batch), and separated during analysis based at least in part on the barcode. In some examples, a barcode may be identified accurately even after mutation, insertion, or deletion of one or more nucleotides in the barcode sequence (e.g., the mutation, insertion, or deletion of at least 1, 2, 3, 4, 5, 6, 7, 8, 9, 10, or more nucleotides). A plurality of polynucleotides from the same sample may have the same barcode. Alternatively, the plurality of polynucleotides from the same sample may have different barcodes. A first barcode may differ from a second barcode by at least three nucleotide positions, such as at least 3, 4, 5, 6, 7, 8, 9, 10, or more nucleotide positions. A plurality of barcodes may be represented in a pool of samples, each sample comprising polynucleotides comprising one or more barcodes that differ from the barcodes contained in the polynucleotides derived from the other samples in the pool. Samples of polynucleotides comprising one or more barcodes can be pooled based on the barcode sequences to which they are joined, such that all four of the nucleotide bases A, G, C, and T are approximately evenly represented at one or more positions along each barcode in the pool (such as at 1, 2, 3, 4, 5, 6, 7, 8, or more positions, or all positions of the barcode). In some examples, the methods of the present disclosure may comprise identifying the sample from which a target polynucleotide is derived based on a barcode sequence to which the target polynucleotide is joined. The barcode may comprise a nucleic acid sequence that when joined to a target polynucleotide may serve as an identifier of the sample from which the target polynucleotide was derived. In an example, an oligonucleotide primer (e.g., an amplification primer) may comprise one or more barcodes. In another example, a nucleic acid molecule may be coupled (e.g., ligated) to an adaptor nucleic acid (e.g., for circularization), and the adaptor nucleic acid may comprise one or more barcodes.

The term “sample,” as used herein, generally refers to any sample that may include one or more constituents (e.g., nucleic acid molecules) for processing or analysis. The sample may be a biological sample. The sample may be a cellular or tissue sample. The sample may be a cell-free sample, such as blood (e.g., whole blood), plasma, serum, sweat, saliva, or urine. The sample may be obtained in vivo or cultured in vitro.

The term “subject,” as used herein, generally refers to an individual or entity from which a sample is derived, such as, for example, a vertebrate (e.g., a mammal, such as a human) or an invertebrate. A mammal may be a murine, simian, human, farm animal (e.g., cow, goat, pig, or chicken), or a pet (e.g., cat or dog). The subject may be a plant. The subject may be a patient. The subject may be asymptomatic with respect to a disease (e.g., cancer). Alternatively, the subject may be symptomatic with respect to the disease.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### I. Systems and Methods for Analysis and Detection of a Target Molecule

In an aspect, the present disclosure provides a system for analyzing or identifying a target molecule. The system may comprise a sensor configured to detect one or more signals indicative of an impedance or impedance change in the sensor when at least a portion of the target molecule is bound by or in proximity to at least a portion of the sensor. The one or more signals may be usable to analyze or identify the target molecule.

The system may comprise at least one of the sensor disclosed herein. The system may comprise at least 1, 2, 3, 4, 5, 6, 7, 8, 9, 10, 20, 30, 40, 50, 60, 70, 80, 90, 100, 200, 300, 400, 500, 600, 700, 800, 900, 1,000 or more sensors. The system may comprise at most 1,000, 900, 800, 700, 600, 500, 400, 300, 200, 100, 90, 80, 70, 60, 50, 40, 30, 20, 10, 9, 8, 7, 6, 5, 4, 3, 2, or less sensor(s).

A detected signal indicative of an impedance or impedance change in the sensor induced by the target molecule maybe a single measurement. Alternatively, the detected signal may be a median or average of a plurality of measurements.

When detecting the one or more signals indicative of the impedance or impedance change in the sensor, at least a portion of the target molecule may be bound to a binding moiety of the sensor. The binding moiety may be configured to bind the at least the portion of the target molecule (e.g., a nucleotide, an amino acid, a small molecule, an ion, etc.). The sensor disclosed herein may comprise at least one binding moiety. The sensor may comprise at least 1, 2, 3, 4, 5, 6, 7, 8, 9, 10, 20, 30, 40, 50, 60, 70, 80, 90, 100, 200, 300, 400, 500, 600, 700, 800, 900, 1,000 or more binding moieties. The sensor may comprise at most 1,000, 900, 800, 700, 600, 500, 400, 300, 200, 100, 90, 80, 70, 60, 50, 40, 30, 20, 10, 9, 8, 7, 6, 5, 4, 3, 2, or less sensor(s).

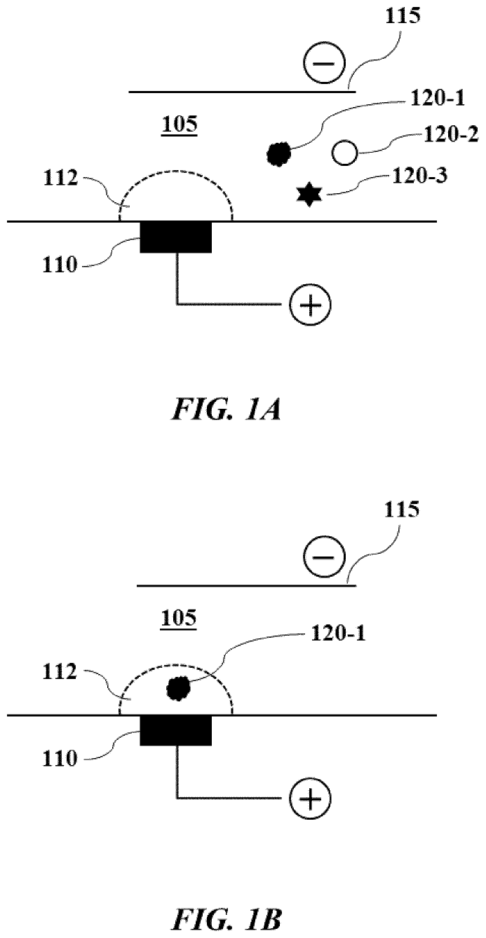

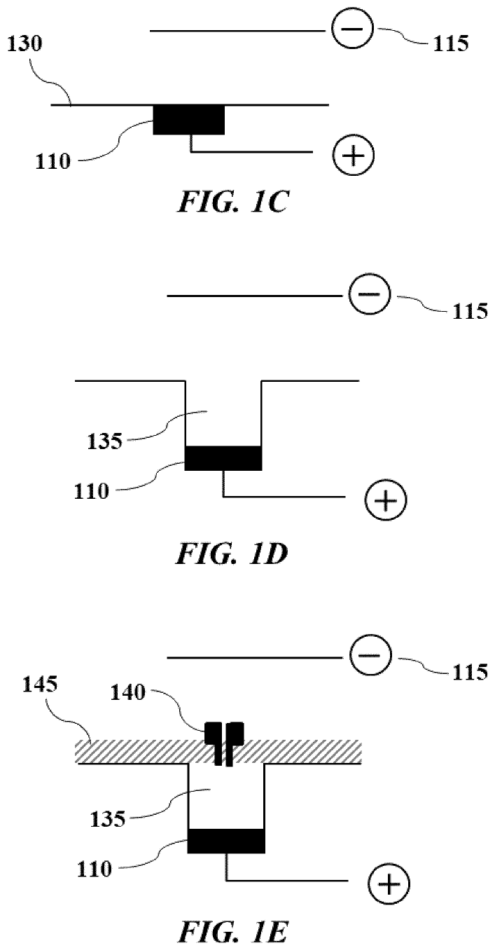

FIGS. 1A through 1E schematically illustrate examples of the system comprising the sensor of the present disclosure, for example, for assessing (e.g., analyzing, identifying, quantifying, ranking, etc.) one or more target molecules. Referring to FIG. 1A, the sensor may be immersed in a liquid 105 (e.g., water or buffer, such as saline, high electrolyte buffer, low electrolyte buffer, etc.). The sensor may comprise a sensing electrode 110 that is in electrical communication with a reference electrode 115. The sensor may be configured to detect or measure one or more LCR (Inductance (L), Capacitance (C), and Resistance (R)) properties of a volume 112 of the liquid 105, which volume 112 is near or adjacent to the sensing electrode 110. The target molecules (labelled as 102-1, 120-2, and 120-3) may be present or floating in the liquid. The target molecule may or may not be coupled to a prove (e.g., a fluorescent tag or a redox species). Referring to FIG. 1B, when at least a portion of any of the target molecules (e.g., target molecule 120-1) is adjacent to or within the volume 112 of the liquid 105, the sensor may detect the one or more signals indicative of an impedance or impedance change in the volume 112.

In some cases, an impedance or impedance change may be detected by applying a constant voltage (e.g., a sinusoidal voltage perturbation) while measuring a current (e.g., a change in the current). An impedance value (Z) may be measured by a value of the applied voltage (V) divided by a value of the measured current (I).

The detected impedance or impedance change, e.g., between the sensing electrode 110 and the reference electrode 115, may be at least about 1 micro-ohm, 2 micro-ohms, 3 micro-ohms, 4 micro-ohms, 5 micro-ohms, 6 micro-ohms, 7 micro-ohms, 8 micro-ohms, 9 micro-ohms, 10 micro-ohms, 20 micro-ohms, 30 micro-ohms, 40 micro-ohms, 50 micro-ohms, 60 micro-ohms, 70 micro-ohms, 80 micro-ohms, 90 micro-ohms, 100 micro-ohms, 200 micro-ohms, 300 micro-ohms, 400 micro-ohms, 500 micro-ohms, 600 micro-ohms, 700 micro-ohms, 800 micro-ohms, 900 micro-ohms, 1 milli-ohm, 2 milli-ohms, 3 milli-ohms, 4 milli-ohms, 5 milli-ohms, 6 milli-ohms, 7 milli-ohms, 8 milli-ohms, 9 milli-ohms, 10 milli-ohms, 20 milli-ohms, 30 milli-ohms, 40 milli-ohms, 50 milli-ohms, 60 milli-ohms, 70 milli-ohms, 80 milli-ohms, 90 milli-ohms, 100 milli-ohms, 200 milli-ohms, 300 milli-ohms, 400 milli-ohms, 500 milli-ohms, 600 milli-ohms, 700 milli-ohms, 800 milli-ohms, 900 milli-ohms, 1 ohm, 2 ohms, 3 ohms, 4 ohms, 5 ohms, 6 ohms, 7 ohms, 8 ohms, 9 ohms, 10 ohms, 20 ohms, 30 ohms, 40 ohms, 50 ohms, 60 ohms, 70 ohms, 80 ohms, 90 ohms, 100 ohms, 200 ohms, 300 ohms, 400 ohms, 500 ohms, 600 ohms, 700 ohms, 800 ohms, 900 ohms, 1 kilo-ohm, 2 kilo-ohms, 3 kilo-ohms, 4 kilo-ohms, 5 kilo-ohms, 6 kilo-ohms, 7 kilo-ohms, 8 kilo-ohms, 9 kilo-ohms, 10 kilo-ohms, 20 kilo-ohms, 30 kilo-ohms, 40 kilo-ohms, 50 kilo-ohms, 60 kilo-ohms, 70 kilo-ohms, 80 kilo-ohms, 90 kilo-ohms, 100 kilo-ohms, 200 kilo-ohms, 300 kilo-ohms, 400 kilo-ohms, 500 kilo-ohms, 600 kilo-ohms, 700 kilo-ohms, 800 kilo-ohms, 900 kilo-ohms, 1,000 kilo-ohms or more. The detected impedance or impedance change, e.g., between the sensing electrode 110 and the reference electrode 115, may be at most about 1,000 kilo-ohms, 900 kilo-ohms, 800 kilo-ohms, 700 kilo-ohms, 600 kilo-ohms, 500 kilo-ohms, 400 kilo-ohms, 300 kilo-ohms, 200 kilo-ohms, 100 kilo-ohms, 90 kilo-ohms, 80 kilo-ohms, 70 kilo-ohms, 60 kilo-ohms, 50 kilo-ohms, 40 kilo-ohms, 30 kilo-ohms, 20 kilo-ohms, 10 kilo-ohms, 9 kilo-ohms, 8 kilo-ohms, 7 kilo-ohms, 6 kilo-ohms, 5 kilo-ohms, 4 kilo-ohms, 3 kilo-ohms, 2 kilo-ohms, 1 kilo-ohm, 900 ohms, 800 ohms, 700 ohms, 600 ohms, 500 ohms, 400 ohms, 300 ohms, 200 ohms, 100 ohms, 90 ohms, 80 ohms, 70 ohms, 60 ohms, 50 ohms, 40 ohms, 30 ohms, 20 ohms, 10 ohms, 9 ohms, 8 ohms, 7 ohms, 6 ohms, 5 ohms, 4 ohms, 3 ohms, 2 ohms, 1 ohm, 900 milli-ohms, 800 milli-ohms, 700 milli-ohms, 600 milli-ohms, 500 milli-ohms, 400 milli-ohms, 300 milli-ohms, 200 milli-ohms, 100 milli-ohms, 90 milli-ohms, 80 milli-ohms, 70 milli-ohms, 60 milli-ohms, 50 milli-ohms, 40 milli-ohms, 30 milli-ohms, 20 milli-ohms, 10 milli-ohms, 9 milli-ohms, 8 milli-ohms, 7 milli-ohms, 6 milli-ohms, 5 milli-ohms, 4 milli-ohms, 3 milli-ohms, 2 milli-ohms, 1 milli-ohm, 900 micro-ohms, 800 micro-ohms, 700 micro-ohms, 600 micro-ohms, 500 micro-ohms, 400 micro-ohms, 300 micro-ohms, 200 micro-ohms, 100 micro-ohms, 90 micro-ohms, 80 micro-ohms, 70 micro-ohms, 60 micro-ohms, 50 micro-ohms, 40 micro-ohms, 30 micro-ohms, 20 micro-ohms, 10 micro-ohms, 9 micro-ohms, 8 micro-ohms, 7 micro-ohms, 6 micro-ohms, 5 micro-ohms, 4 micro-ohms, 3 micro-ohms, 2 micro-ohms, 1 micro-ohm, or less.

The detected impedance or impedance change, e.g., between the sensing electrode 110 and the reference electrode 115, may be a measurement (e.g., a single measurement, a plurality of measurements to yield an average value of the plurality of measurements) taken over a period of at least about 1 nanosecond, 2 nanoseconds, 3 nanoseconds, 4 nanoseconds, 5 nanoseconds, 6 nanoseconds, 7 nanoseconds, 8 nanoseconds, 9 nanoseconds, 10 nanoseconds, 20 nanoseconds, 30 nanoseconds, 40 nanoseconds, 50 nanoseconds, 60 nanoseconds, 70 nanoseconds, 80 nanoseconds, 90 nanoseconds, 100 nanoseconds, 200 nanoseconds, 300 nanoseconds, 400 nanoseconds, 500 nanoseconds, 600 nanoseconds, 700 nanoseconds, 800 nanoseconds, 900 nanoseconds, 1 microsecond, 2 microseconds, 3 microseconds, 4 microseconds, 5 microseconds, 6 microseconds, 7 microseconds, 8 microseconds, 9 microseconds, 10 microseconds, 20 microseconds, 30 microseconds, 40 microseconds, 50 microseconds, 60 microseconds, 70 microseconds, 80 microseconds, 90 microseconds, 100 microseconds, 200 microseconds, 300 microseconds, 400 microseconds, 500 microseconds, 600 microseconds, 700 microseconds, 800 microseconds, 900 microseconds, 1 millisecond, 2 milliseconds, 3 milliseconds, 4 milliseconds, 5 milliseconds, 6 milliseconds, 7 milliseconds, 8 milliseconds, 9 milliseconds, 10 milliseconds, 20 milliseconds, 30 milliseconds, 40 milliseconds, 50 milliseconds, 60 milliseconds, 70 milliseconds, 80 milliseconds, 90 milliseconds, 100 milliseconds, 200 milliseconds, 300 milliseconds, 400 milliseconds, 500 milliseconds, 600 milliseconds, 700 milliseconds, 800 milliseconds, 900 milliseconds, 1 second, 2 seconds, 3 seconds, 4 seconds, 5 seconds, 6 seconds, 7 seconds, 8 seconds, 9 seconds, 10 seconds, or more. The detected impedance or impedance change, e.g., between the sensing electrode 110 and the reference electrode 115, may be a measurement (e.g., a single measurement, a plurality of measurements to yield an average value of the plurality of measurements) taken over a period of at most about 10 seconds, 9 seconds, 8 seconds, 7 seconds, 6 seconds, 5 seconds, 4 seconds, 3 seconds, 2 seconds, 1 second, 900 milliseconds, 800 milliseconds, 700 milliseconds, 600 milliseconds, 500 milliseconds, 400 milliseconds, 300 milliseconds, 200 milliseconds, 100 milliseconds, 90 milliseconds, 80 milliseconds, 70 milliseconds, 60 milliseconds, 50 milliseconds, 40 milliseconds, 30 milliseconds, 20 milliseconds, 10 milliseconds, 9 milliseconds, 8 milliseconds, 7 milliseconds, 6 milliseconds, 5 milliseconds, 4 milliseconds, 3 milliseconds, 2 milliseconds, 1 millisecond, 900 microseconds, 800 microseconds, 700 microseconds, 600 microseconds, 500 microseconds, 400 microseconds, 300 microseconds, 200 microseconds, 100 microseconds, 90 microseconds, 80 microseconds, 70 microseconds, 60 microseconds, 50 microseconds, 40 microseconds, 30 microseconds, 20 microseconds, 10 microseconds, 9 microseconds, 8 microseconds, 7 microseconds, 6 microseconds, 5 microseconds, 4 microseconds, 3 microseconds, 2 microseconds, 1 microsecond, 900 nanoseconds, 800 nanoseconds, 700 nanoseconds, 600 nanoseconds, 500 nanoseconds, 400 nanoseconds, 300 nanoseconds, 200 nanoseconds, 100 nanoseconds, 90 nanoseconds, 80 nanoseconds, 70 nanoseconds, 60 nanoseconds, 50 nanoseconds, 40 nanoseconds, 30 nanoseconds, 20 nanoseconds, 10 nanoseconds, 9 nanoseconds, 8 nanoseconds, 7 nanoseconds, 6 nanoseconds, 5 nanoseconds, 4 nanoseconds, 3 nanoseconds, 2 nanoseconds, 1 nanosecond, or less.

Referring to FIGS. 1A and 1B, an electric field may be applied between the sensing electrode and the reference electrode. In some cases, a function generator may be operatively coupled to the sensor and apply a voltage (e.g., a small amplitude sine wave voltage), causing a current in the sensor's circuit. Following, the current, as well as the amplitude and phase (of the alternating current (i.e., a.c.) component) may be measured. Such value may be converted into the one or more signal indicative of the impedance or impedance change in the sensor. The measurement may be repeated at a series of different voltages (or frequencies), thus building up an impedance spectrum of the sensor. In some cases, a LCR meter may be operatively coupled to the sensor to measure one or more of the LCR signals or signatures of the sensor when the target molecule is in contact with (e.g., directly or indirectly) or in the vicinity of the sensing electrode.

Referring to FIG. 1C, the sensing electrode 110 of the sensor may be disposed adjacent to a roughly flat or planar surface 130. Referring to FIG. 1D, the sensing electrode 110 of the sensor may be disposed with in a well 135, e.g., at a bottom portion of the well 135. The well 135 may be a closed-bottom well. In some cases, at least a portion of a surface of the well (e.g., a portion of the surface of the well that is not covered by the sensing electrode) may be covered by a dielectric material (e.g., an insulating material). Alternatively, a surface of the well may not be covered by a dielectric material. The well may help localize or trap the target molecule inside the well for a longer duration of time and/or with a higher binding affinity to improve accuracy and sensitivity of the sensor. In some cases, (i) a first target molecule may be trapped by the well for measurement of a first signal indicative of impedance or impedance change in the sensor, and (ii) a subsequent target molecule may be trapped by the well for measurement of an additional signal indicative of impedance or impedance change in the sensor. During (ii), the first target molecule may remain trapped within the well. For example, the target molecule may be bound to a substrate that is disposed within the well. Alternatively, the first target molecule may not be trapped in the well during (ii). For example, the first target molecule may exit the well prior to (ii). Referring to FIG. 1E, the sensing electrode 110 of the sensor may be disposed beneath a nanopore (e.g., a protein nanopore or a solid state nanopore). In this example, the sensing electrode 110 may be disposed beneath a protein nanopore 140 that is embedded within a lipid bilayer 145. The lipid bilayer 145 may or may not have capacitance. The nanopore 140 may be configured to function as a charge and/or size filter, thereby controlling what goes through the nanopore 140 and into the well 135 to induce a change in the LCR properties of the sensor. A size of the nanopore 145 may be regulatable. An overall charge of the nanopore 145 may be regulatable. In an example, applying a positive electrical signal to the nanopore 145 may help filter out positively charged molecules and only allow negatively charged molecules to enter and interact with the sensing electrode 110. In some cases, the sensor as shown in FIG. 1E may detect one or more signals indicative of an impedance or impedance change within the sensor subsequent to (i) penetration of the target molecule (or a tag initially coupled to the target molecule) through the nanopore 140 and (ii) coupling (e.g., direct or indirect) of the penetrated target molecule (or the tag) to the sensing electrode 110. Such one or more signals may not be detected while the target molecule (or the tag) is within the nanopore 140. In an example, at least a portion of the target molecule (or the tag) may be coupled to the sensing electrode (e.g., via a linker, such as a conducting material) for detection of the one or more signals. In another example, at least a portion of the target molecule (or the tag) may be in proximity to the conducting material for detection of the one or more signals.

In another aspect, the present disclosure provides a system for analyzing or identifying a target molecule. The system may comprise a sensor comprising a sensing electrode and a reference electrode in electrical communication with one another. The sensor may comprise a dielectric material coupled to the sensing electrode and covering a first portion of a surface of the sensing electrode. The sensor may comprise a conducting material coupled to the sensing electrode and covering a second portion of the surface of the sensing electrode. The sensor may comprise a binding unit coupled to the conducting material, wherein the binding unit is configured to bind the target molecule. The sensor may be configured to detect one or more signals indicative of an impedance or impedance change in the sensor when at least a portion of the target molecule is bound by the binding unit. The one or more signals may be usable to analyze or identify the target molecule. The conducting material may be a bond (e.g., a chemical bond) or may comprise a linking unit (e.g., a nanorod, a peptide, a small molecule, etc.) of any desired dimension (e.g., length, cross-sectional diameter or area, volume, etc.). In an alternative aspect, the binding unit may be directly coupled to the sensing electrode. Yet in a different aspect, the binding unit may be coupled to at least a portion of the dielectric material that is coupled to the sensing electrode.

The one or more signals may be indicative of (i) electrical resistance or a change thereof in the sensor, (ii) electrical capacitance or a change thereof in the sensor, or (ii) an electrical inductance or a change thereof in the sensor. The one or more signals may be indicative of at least two of: (i) electrical resistance or a change thereof in the sensor, (ii) electrical capacitance or a change thereof in the sensor, and (ii) an electrical inductance or a change thereof in the sensor. The one or more signals may be indicative of (i) electrical resistance or a change thereof in the sensor, (ii) electrical capacitance or a change thereof in the sensor, or (ii) an electrical inductance and a change thereof in the sensor.

The one or more signals may be current or voltage. The one or more signals may be current and voltage. The one or more signals may not be tunneling current.

The first portion of the sensing electrode that is covered by the dielectric material may be at least 50 percent (%) of the surface of the sensing electrode. In some cases, the first portion of the sensing electrode may be at least 50%, 55%, 60%, 65%, 70%, 75%, 80%, 85%, 90%, 95%, 99%, or more of the surface of the sensing electrode. In some cases, the first portion of the sensing electrode may be at most 100%, 95%, 90%, 85%, 80%, 75%, 70%, 65%, 60%, 55%, 50%, or less of the surface of the sensing electrode.

The second portion of the sensing electrode may be at most 50% of the surface of the sensing electrode. In some cases, the second portion of the sensing electrode may be at most 50%, 45%, 0%, 35%, 0%, 25%, 20%, 15%, 10%, 9%, 8%, 7%, 6%, 5%, 4%, 3%, 2%, 1%, or less of the surface of the sensing electrode. In some cases, the second portion of the sensing electrode may be at least 1%, 2%, 3%, 4%, 5%, %, 7%, 8%, 9%, 10%, 15%, 20%, 25%, 30%, 35%, 40%, 45%, 50%, or more of the surface of the sensing electrode.

An average cross-sectional dimension (or a surface area that is coupled to the dielectric material and the conducting material) of the sensing electrode may be no more than 100-fold greater than an average size of the target molecule. In some cases, the average cross-sectional dimension of the sensing electrode may be at most 100-fold, 90-fold, 80-fold, 70-fold, 60-fold, 50-fold, 40-fold, 30-fold, 25-fold, 20-fold, 15-fold, 10-fold, 9-fold, 8-fold, 7-fold, 6-fold, 5-fold, 4-fold, 3-fold, 2-fold, 1-fold, 0.5-fold, or 0.1-fold greater than the average size of the target molecule. In some cases, the average cross-sectional dimension of the sensing electrode may be at least 0.1-fold, 0.5-fold, 1-fold, 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, or 100-fold greater than the average size of the target molecule. In some cases, the average cross-sectional dimension of the sensing electrode may be no more than at least 0.1 nanometers (nm), 0.5 nm, 1 nm, 2 nm, 3 nm, 4 nm, 5 nm, 10 nm, 50 nm, 100 nm, 500 nm, 1,000 nm, 5,000 nm, 10,000 nm, or more than the average size of the target molecule. In some cases, the average cross-sectional dimension of the sensing electrode may be at most 10,000 nm, 5,000 nm, 1,000 nm, 500 nm, 100 nm, 50 nm, 10 nm, 5 nm, 4 nm, 3 nm, 2 nm, 1 nm, 0.5 nm, 0.1 nm, or less than the average size of the target molecule.

Alternatively, the average cross-sectional dimension of the sensing electrode may be smaller than the average size of the target molecule. In some cases, the average cross-sectional dimension of the sensing electrode may be at least 1%, 2%, 3%, 4%, 5%, 6%, 7%, 8%, 9%, 10%, 15%, 20%, 25%, 30%, 40%, or 50% smaller than the average size of the target molecule. In some cases, the average cross-sectional dimension of the sensing electrode may be at most 50%, 40%, 30%, 20%, 15%, 10%, 9%, 8%, 7%, 6%, 5%, 4%, 3%, 2%, or 1% smaller than the average size of the target molecule.

The area of the second portion of the surface of the sensing electrode may be no more than 500 Angstrom squared (Å²), 100 Å², 50 Å²,10 Å², 9 Å², 8 Å², 7 Å², 6 Å², 5 Å², 4 Å², 3 Å², 2 Å², 1 Å², or less. In some cases, a cross-sectional dimension or the diameter of the second portion of the surface of the sensing electrode may be approximately equal to a diameter (e.g., twice that of the Van der Waals radius) of an atom of the conducting material.

The dielectric material may be a solid layer (e.g., a solid metal or semi-conducting material) or a self-assembled monolayer (SAM).

The conducting material may be a single molecule (e.g., a single conducting polymeric chain). In some cases, one or more features of atomic force microscopy (AFM) (e.g., a piezoelectric cantilever probe of the AFM) to couple the single molecule to a specific (or random) position within the surface of the sensing electrode. Alternatively, the conducting material may be a plurality of molecules (e.g., a plurality of identical and/or different conducting polymeric chains). The conducting material may be comprised of at least 1, 5, 10, 50, 100, 500, 1,000, 5,000, 10,000, 50,000, 100,000, or more molecules. The conducting material may be comprised of at most 100,000, 50,000, 10,000, 5,000, 1,000, 500, 100, 50, 10, 5, or 1 molecule.

In some cases, a substrate may be bound to the binding unit (e.g., a nuclease or a variant thereof) of the sensor of the present disclosure, and the binding unit or an additional element (e.g., an additional enzyme) may be configured to cleave at least a portion of the substrate. The sensor may be configured to (i) detect a difference in the one or more signals indicative of impedance change in the sensor following such cleavage, and (ii) identify what has been cleaved off based on analyzing the one or more signals.

The binding unit may be an enzyme, an antibody, an aptamer, a non-biological material (e.g., a synthetic polymer), a functional fragment thereof, a functional variant thereof, or a combination thereof. The enzyme may be selected from the group consisting of a polymerase, a nuclease (e.g., double-stranded nuclease), nickase, transcriptional activator, transcriptional repressor, nucleic acid methylation enzyme, nucleic acid demethylation enzyme, and recombinase. The antibody may be a whole antibody or antigen-binding fragment thereof, such as an scFv, a Fab fragment, a VHH domain, or a VH domain of a heavy-chain only antibody. The antibody may be mono-specific or multi-specific (e.g., bi-specific, tri-specific, etc.). The antibody may be mono-valent or multi-valent (e.g., bi-valent, tri-valent, etc.).

The binding unit may be directly coupled to the conducting material, e.g., covalently or non-covalently attached to the conducting material. Alternatively, the binding unit may be indirectly coupled to the sensing electrode, e.g., via a linker that binds the conducting material on one side and the binding unit on the other side. In such cases, the linker may also be a conducting linker as to minimize interference of the sensing capabilities of the sensor. Alternatively, the linker may not be conducting.

The target molecule may be selected from the group consisting of a small molecule, a nucleotide, a polynucleotide, an amino acid, a peptide, a polypeptide, a variant thereof, and a combination thereof.

The target molecule may comprise one of more tags. The target molecule may comprise at least 1, 2, 3, 4, 5, or more tags. The target molecule may comprise at most 5, 4, 3, 2, or 1 tag. The tag may be configured to induce a change in the one or more signals of the sensor. In some cases, a plurality of types of tags may be used for a plurality of target molecules, and one or more signals of each of the target molecules with their respective tag may be approximately distinguishable from the others. In some cases, one or more features or characteristics of the target molecule (e.g., size, shape, charge, vibration, movement within in the fluid, etc.) may further affect the one or more signals of the sensors, thereby further rendering the one or more signals of each of the target molecules with their respective tag more distinguishable from the others. In some cases, a tag of the present disclosure may be an impedance tag. The impedance tag may be configured to elicit a change in detected impedance in the sensor, e.g., between a sensing electrode and a reference electrode. Examples of the impedance tag can include, but are not limited to, organic compounds, organometallic compounds, nanoparticles, metals, functional variants thereof, and combinations thereof.

Alternatively, the target molecule may not comprise any tag. In such cases, one or more features or characteristics of the target molecule (e.g., size, shape, charge, vibration, movement within in the fluid, etc.) may further affect the one or more signals of the sensors, thereby further rendering the one or more signals of each of the target molecules with their respective tag more distinguishable from the others. In an example, the binding unit may be a protein in a biological sample, such as blood, plasma, or urine. The binding unit may be an enzyme (e.g., polymerase configure to perform rolling circle amplification (RCA)) that is bound to a circular nucleic acid for RCA. The target moiety may be a nucleobase without any fluorescent or redox species tag, but the sensitivity of the sensor of the present disclosure may be capable of analyzing the one or more signals indicative of an impedance or impedance change in the sensor when the nucleotide is bound to the circular nucleic acid by the polymerase.

Size, shape, and/or resolution of one or more components of the sensor of the present disclosure may be defined or limited by resolutions of techniques such as photolithography, etching, nanoimprinting, size of bio nanopores, etc.

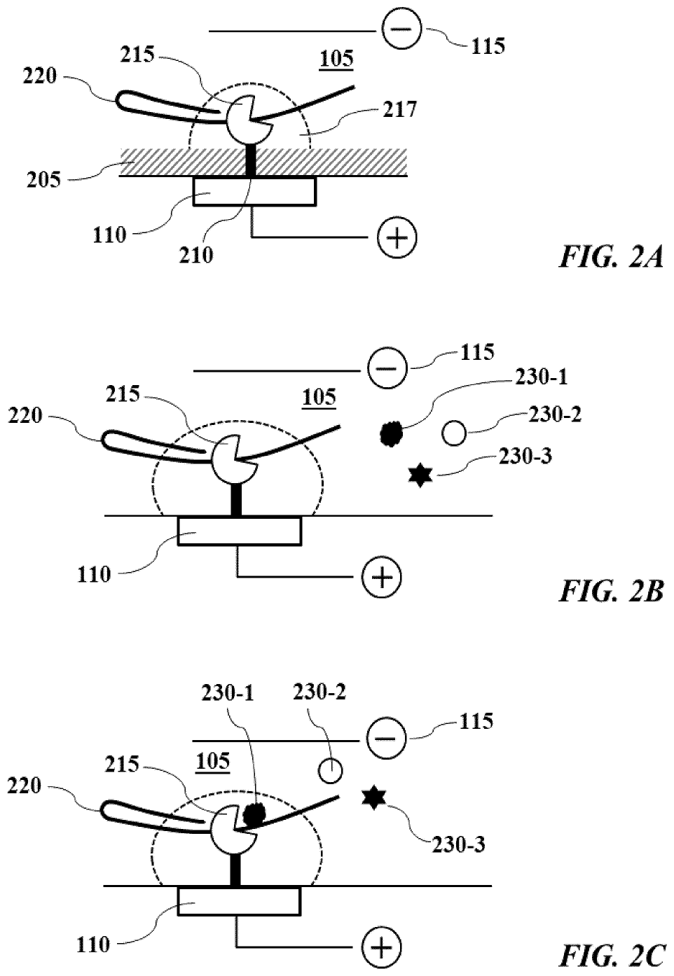

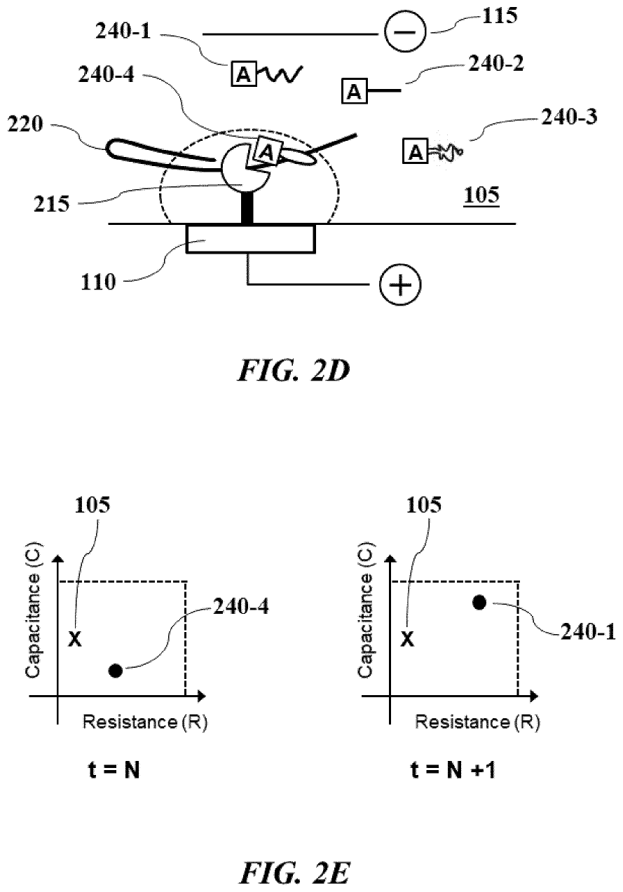

FIGS. 2A through 2E schematically illustrate examples of the system comprising the sensor of the present disclosure, for example, for assessing (e.g., analyzing, identifying, quantifying, ranking, etc.) one or more target molecules. Referring to FIG. 2A, the sensor may be immersed in the liquid 105 (e.g., water or buffer, such as saline, high electrolyte buffer, low electrolyte buffer, etc.). The sensor may comprise a sensing electrode 110 that is in electrical communication with a reference electrode 115. The sensor may comprise a dielectric material (e.g., SAM) 205 coupled to at least a top surface of the sensing electrode 110. The SAM 205 may cover most of the top surface of the sensing electrode 110. Additionally, a conducting material 210 (e.g., one or more conducting polymeric chains) may be coupled to the top surface of the sensing electrode 110. The conducting material 210 may be embedded within the “sea” of the SAM 205. Also, a binding moiety 215 may be coupled to the conducting material 210 at a side (or end) opposite the sensing electrode 110. The sensor may be configured to detect or measure one or more LCR properties or a change thereof within a volume 217 of the liquid 105, which volume 217 is near or adjacent to the binding unit 215. In this example, the binding unit 215 may be an enzyme (e.g. a polymerase), and the enzyme may be configured to add one or more nucleotides to a polynucleotide substrate 220.

A thickness of the dielectric material (e.g., the SAM 205 as shown in FIG. 2A) that is coupled to at least a portion of a surface of a sensing electrode may be about at least 0.1 nm, 0.5 nm, 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. A thickness of the dielectric material that is coupled to at least a portion of a surface of a sensing electrode may be at most about, 000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 9 nm, 8 nm, 7 nm, 6 nm, 5 nm, 4 nm, 3 nm, 2 nm, 1 nm, 0.5 nm, 0.1 nm, or less.

Referring to FIG. 2B, the target molecules 230 may be three nucleotide probes (labelled as 1, 2, and 3) floating in the liquid 105. Referring to FIG. 2C, the binding unit 215 (e.g., the polymerase) that is coupled to the sensing electrode 110 and embedded within the SAM (not shown) may interact with a target nucleotide probe 230-1 and couple the target nucleotide probe to the nucleotide substrate 220. The sensor may measure the one or more signals indicative of an impedance or impedance change in the sensor when (i) the nucleotide probe 230-1 comprising the tag is bound to the binding unit 215 and/or (ii) the binding unit 215 or an additional enzyme operatively coupled to the binding unit 215 cleaves the tag from the nucleotide probe 230-1. In some cases, the impedance or the impedance change as measured may be a bulk impedance or impedance change. In some examples, the detected impedance or impedance change is not within the Debye (or double) layer of the electrode, e.g., the sensor electrode 110 as shown in FIG. 2C. In an example, the detected impedance or impedance change may be outside of the Debye layer of the electrode. In some examples, the detected impedance or impedance change is not within the Debye (or double) layer of the dielectric material (e.g., the SAM 205 as shown in FIG. 2A) that is coupled to the sensing electrode. In an example, the detected impedance or impedance change may be outside of the Debye layer of the dielectric material.

Referring to FIG. 2D, the target molecules 230 may be four nucleotide probes (labelled as 240-1, 240-2, 240-3, and 240-4) floating in the liquid 105. Here, the nucleotide probes have identical nucleotide base (e.g., adenine, or A), but with different tags, as drawn by different shaped tags. At a given position along the length of the nucleotide substrate 220 (i.e., t=N), the binding unit 215 (e.g., the polymerase) that is coupled to the sensing electrode 110 and embedded within the SAM (not shown) may interact with a target nucleotide probe 240-4 (e.g., by a random selection based on free movement of the nucleotide probes in the liquid 105) and couple the target nucleotide probe 240-4 to the nucleotide substrate 220. Following, the sensor may detect the one or more signals (e.g., capacitance vs resistance plot) indicative of an impedance or impedance change in the sensor due to the addition of the nucleotide probe 240-4 to the substrate 220. Similarly, when the substrate again calls out of the same nucleobase as before for the next position of the nucleotide substrate 220 (i.e., t=N+1), the binding unit 215 may interact with another target nucleotide probe 240-1 (e.g., by a random selection) and couple the target nucleotide probe 240-1 to the nucleotide substrate 220. Following, the sensor may detect the one or more signals (e.g., capacitance vs resistance plot) indicative of an impedance or impedance change in the sensor due to the addition of the nucleotide probe 240-1 to the substrate 220.

In some cases, the sensor may be configured to detect one or more signals indicative of the impedance or impedance change, e.g., between the sensing electrode 110 and the reference electrode 115, when at least a portion of a target molecule (and/or a tag coupled to the target molecule) is bound (e.g., directly, or indirectly via the binding unit 215 and the conducting material 210) to at least a portion of the sensor, e.g., the sensing electrode 110. Alternatively, the sensor may be configured to detect one or more signals indicative of the impedance or impedance change, e.g., between the sensing electrode 110 and the reference electrode 115, when at least a portion of a target molecule (and/or a tag coupled to the target molecule) is not bound but in proximity to at least a portion of the sensor, e.g., the sensing electrode 110.

The sensor of the present disclosure may be configured to detect more signals indicative of the impedance or impedance change, e.g., between the sensing electrode and the reference electrode, when a distance between (i) at least a portion of a target molecule (and/or a tag coupled to the target molecule) and (ii) the sensing electrode is at least about 0.1 nm, 0.5 nm, 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1,000 μm, or more. The sensor as disclosed herein may be configured to detect more signals indicative of the impedance or impedance change, e.g., between the sensing electrode and the reference electrode, when a distance between (i) at least a portion of a target molecule (and/or a tag coupled to the target molecule) and (ii) the sensing electrode is at most about 1,000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 9 nm, 8 nm, 7 nm, 6 nm, 5 nm, 4 nm, 3 nm, 2 nm, 1 nm, 0.5 nm, 0.1 nm, or less.

The sensor of the present disclosure may be configured to detect more signals indicative of the impedance or impedance change, e.g., between the sensing electrode and the reference electrode, when a target molecule (and/or a tag coupled to the target molecule) is within a predetermined space (e.g., the predetermined volume 217 as shown in, for instance, FIG. 2A) that is near or adjacent to the sensing electrode. The predetermined space may be characterized by having a volume of at least about 0.1 nm², 0.5 nm², 1 nm, 2 nm², 3 nm², 4 nm², 5 nm², 6 nm², 7 nm², 8 nm², 9 nm², 10 nm², 20 nm², 30 nm², 40 nm² 50 nm², 60 nm², 70 nm², 80 nm², 90 nm², 100 nm², 200 nm², 300 nm², 400 nm², 500 nm², 600 nm², 700 nm², 800 nm², 900 nm², 1 μm², 2 μm², 3 μm², 4 μm², 5 μm², 6 μm², 7 μm², 8 μm², 9 μm², 10 μm², 20 μm², 30 μm², 40 μm², 50 μm², 60 μm², 70 μm², 80 μm², 90 μm², 100 μm², 200 μm², 300 μm², 400 μm²′ 500 μm², 600 μm², 700 μm², 800 μm², 900 μm², 1,000 μm², or more. The predetermined space may be characterized by having a volume of at most about 1,000 μm², 900 μm², 800 μm², 700 μm², 600 μm², 500 μm², 400 μm², 300 μm², 200 μm², 100 μm², 90 μm², 80 μm², 70 μm², 60 μm², 50 μm², 40 μm², 30 μm² 20 μm², 10 μm², 9 μm², 8 μm², 7 μm², 6 μm², 5 μm², 4 μm², 3 μm², 2 μm², 1 μm², 900 nm², 800 nm², 700 nm², 600 nm², 500 nm², 400 nm², 300 nm², 200 nm², 100 nm², 90 nm², 80 nm², 70 nm², 60 nm², 50 nm² 40 nm², 30 nm², 20 nm², 10 nm², 9 nm², 8 nm², 7 nm², 6 nm², 5 nm², 4 nm², 3 nm², 2 nm², 1 nm², 0.5 nm² 0.1 nm², or less.

The sensor of the present disclosure may not require at least a portion of a target molecule (and/or a tag coupled to the target molecule) to enter and/or pass through a pore (e.g., a nanopore, such as a protein nanopore or a solid state nanopore) to detect one or more signals indicative of the impedance or impedance change. For example, the sensor may not comprise or may not be operatively coupled to a nanopore, as illustrated in, for instance, in FIGS. 2A-2D. Alternatively, at least a portion of a target molecule (and/or a tag coupled to the target molecule) may enter and/or pass through a pore of a sensor (e.g., a nanopore, such as a protein nanopore or a solid state nanopore) in order for the sensor to detect one or more signals indicative of the impedance or impedance change. For example, the sensor may comprise a nanopore, as illustrated in FIG. 1E.

FIG. 2E shows examples of the capacitance vs resistance plot obtained by the sensor after each of the nucleotide probes 240-4 and 240-1 is added to the substrate 220 by the binding unit 215. A measurement of the impedance signal of the liquid 105 may be shown as background or control. As shown by the plots in FIG. 2E, although being coupled to the same nucleotide base (e.g., adenine), different tags may yield different capacitance vs resistance characteristics, thereby allowing the sensor to resolve sequential addition of two or more of the same nucleobases to the substrate 220. In an example, the sensor of the present disclosure may be able to resolve a difference between (i) a first time (t) indicative of an addition of N^{th} adenine (e.g., 20^{th} adenine) and (ii) a second time indicative of a subsequent addition of (N+1)^{th} adenine (e.g., 21^{st} adenine).

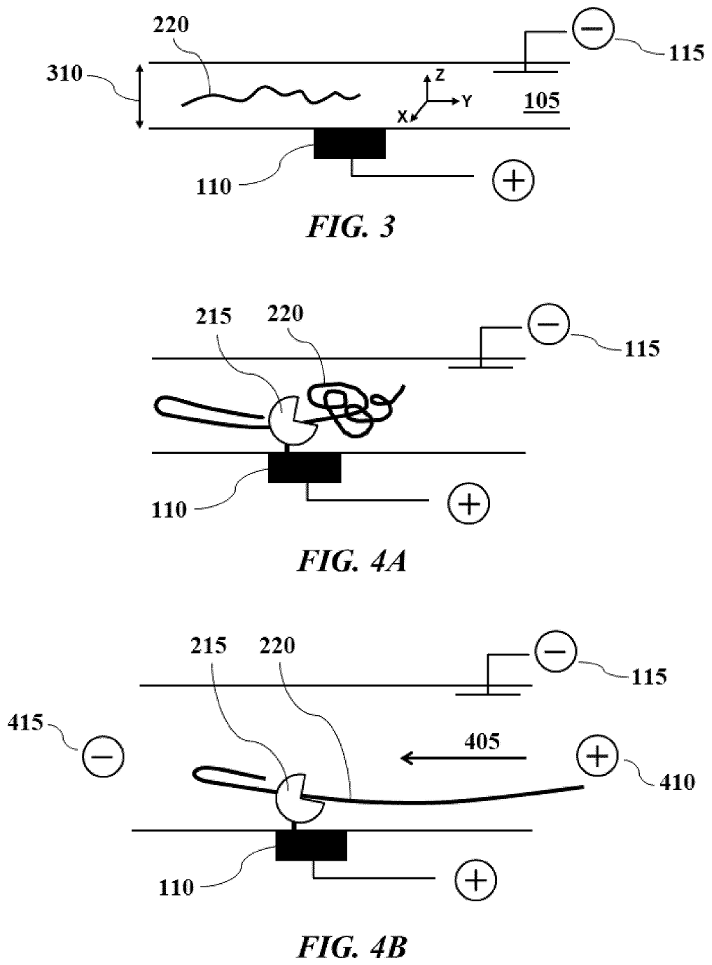

In some embodiments, as shown in FIG. 3, a height 310 of the channel 305 holding the fluid 105 may be designed to inhibit mobility (or diffusivity) of the target molecule along the axis parallel to the height 310 of the channel 305. When effective working area of the sensor (e.g., a dimension of the binding unit, conducting material, or the sensing electrode) is limited, any mobility of the target molecule along an axis perpendicular to the effective working area of the sensor (e.g., the axis parallel to the height 310) may negatively affect measurements of the one or more signals. Thus, by reducing the height 310 of the channel 305 may render the system to be effectively two-dimensional, thereby limiting any effect from a perpendicular motion of the target molecule relative to the sensing electrode 110. The height 310 of the channel may be at most 300 micrometers (μm), 250 μm, 200 μm, 150 μm, 100 μm, 90 μm, 80 μm, 70 μm, 60 μm, 50 μm, 40 μm, 30 μm, 25 μm, 20 μm, 15 μm, 10 μm, 9 μm, 8 μm, 7 μm, 6 μm, 5 μm, 4 μm, 3 μm, 2 μm, 1 μm, or less. The height 310 of the channel may be at least 1 μm, 2 μm, 3 μm, 4 μm, 5 μm, 6 μm, 7 μm, 8 μm, 9 μm, 10 μm, 15 μm, 20 μm, 25 μm, 30 μm, 40 μm, 50 μm, 60 μm, 70 μm, 80 μm, 90 μm, 100 μm, 150 μm, 200 μm, 250 μm, 300 μm, or more.

Referring to FIG. 3, in some cases, the height 310 of the channel 305 may be predetermined (e.g., during fabrication of the sensor). Alternatively, the height 310 of the channel 305 may be adjustable. In some cases, depending on the size of the target molecule or an average size of a population of target molecules, adjusting the height 310 of the channel 305 may be desired. In such cases, one or more switches (e.g., mechanical switches, microfluidic switches), may be disposed on or adjacent to at least a portion of the sensor to vary the height 310 in real time. Examples of the switches may include, but are not limited to, thermally or piezo-electrically driven actuators, motorized actuators, etc.

In some embodiments, the sensing electrode and the reference electrode may provide a first electric field along a first direction. In addition, the system may further comprise additional electric field generator (e.g., an additional set of electrodes in a different circuit) configured to apply a second electric field in a second direction that is different (e.g., substantially perpendicular) to the first direction of the first electric field. A difference between the first direction and the second direction may be at least about 1 degree, 2 degrees, 3 degrees, 4 degrees, 5 degrees, 6 degrees, 7 degrees, 8 degrees, 9 degrees, 10 degrees, 15 degrees, 20 degrees, 30 degrees, 40 degrees, 50 degrees, 60 degrees, 70 degrees, 80 degrees, 90 degrees, 100 degrees, 110 degrees, 120 degrees, 130 degrees, 140 degrees, 150 degrees, 160 degrees, 170 degrees, or more. A difference between the first direction and the second direction may be at most about 180 degrees, 170 degrees, 160 degrees, 150 degrees, 140 degrees, 130 degrees, 120 degrees, 110 degrees, 100 degrees, 90 degrees, 80 degrees, 70 degrees, 60 degrees, 50 degrees, 40 degrees, 30 degrees, 20 degrees, 15 degrees, 10 degrees, 9 degrees, 8 degrees, 7 degrees, 6 degrees, 5 degrees, 4 degrees, 3 degrees, 2 degrees, 1 degree, or less. In an example, the difference between the first direction and the second direction may about 90 degrees (i.e., the first direction and the second direction may be substantially orthogonal to one another). FIG. 4A schematically illustrates a system comprising a sensor, for example, for assessing (e.g., analyzing, identifying, quantifying, ranking, etc.) one or more target molecules, wherein the sensor does not comprise any additional electric field generator. When a polynucleotide substrate 220 is large and folded onto itself when bound to the binding unit 215. Folding of the polynucleotide substrate 220 may render one or more positions of the substrate 220 inaccessible by the binding unit (e.g., polymerase enzyme) to add a target nucleotide, and thus negatively effecting sensitivity and/or accuracy of the sensor. In contrast, FIG. 4B schematically illustrates a different system comprising a sensor and the additional electric field generator configured to apply the additional electric field 405 between the additional set of electrodes 410 and 415, for example, for assessing (e.g., analyzing, identifying, quantifying, ranking, etc.) one or more target molecules. As illustrated, the direction of the electric field 405 may be substantially perpendicular to a surface of the sensing electrode 110. The applied electric field 405 may unfold and/or stretch the substrate 220 along the direction of the electric field 405, thereby reducing folding and overlapping of the substrate 220 by the binding unit 215, conducting material (not shown), and/or the surface of the sensing electrode 110.

The system may comprise at least 1, 2, 3, 4, 5, or more additional electric field generators. The system may comprise at most 5, 4, 3, 2, or 1 additional electric field generator. When comprising a plurality of additional electric field generators, the plurality of additional electric field generators may apply a plurality of electric fields that are along the same or different directions. Alternatively, the system may not comprise any additional electric field generator.

In some embodiments, the target molecules may comprise one or more redox moieties (e.g., as tags), and the sensor of the present disclosure may be configured to measure redox potential (e.g., reduction potential or oxidation potential) of the redox moieties. In an example, the sensor may be configured to measure oxidation potential of an oxidizable functional group of the target molecule. A buffer solution can negatively affect activity of the binding unit (e.g., an enzymatic activity of the binding unit), and thus it may be desirable for some binding unit to reduce a concentration of salt in the solution. However, in some cases, a reduced concentration of salt in the buffer may increase resistance of the system, and thereby reduce sensitivity or accuracy of the sensor. In such cases, utilizing a tag with one or more redox moieties and measuring the redox potential of the target molecule comprising such tag may be advantageous, and improve accuracy.

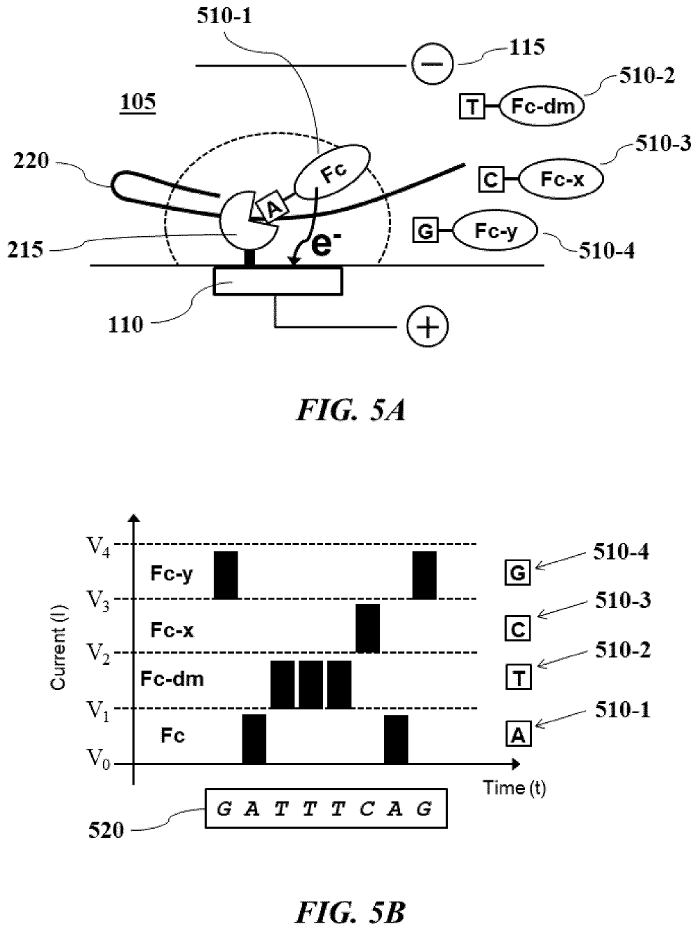

FIG. 5A schematically illustrates an example of a system comprising a sensor that measures oxidation potentials of one or more target molecules, for example, for assessing (e.g., analyzing, identifying, quantifying, ranking, etc.) the one or more target molecules. The sensor may comprise one or more components of the system illustrated in FIG. 2A, e.g., the sensing electrode 110, the reference electrode 115, the liquid 105 (e.g., a salt solution), the binding unit 215 (e.g., a polymerase), and the polynucleotide substrate 220. Here, the target molecules which are to be added to substrate 220 by the binding unit 215 are synthetic nucleotides A, T, C, and G, as denoted by 510-1, 510-2, 510-3, and 510-4. The redox moieties of the target molecules may be ferrocene (Fc) or a series of Fc derivatives, i.e., Fc-x, Fc-γ, and Fc-dm. Fc and its derivatives may have different oxidation potentials, and thus when each target molecule comprising its respective redox tag is added to the substrate 220 by the binding unit 215, application of different voltages (e.g., varied by a few millivolts) may be able to resolve which target molecule has been added to the substrate 220, e.g., by detecting electron transfer between (i) a redox moiety of a target molecule and (ii) the sensing electrode when the target molecule bound to the redox moiety is bound by the binding unit to be added to the substrate 220.

FIG. 5B schematically illustrates a time (t, x-axis) versus current (I, y-axis) plot at varied voltages (indicated as V₀, V₁, V₂, V₃, and V₄ along the y-axis), and current (indicated by square peaks) plot obtained from the sensor of FIG. 5A. A plurality of time versus current plots at varied voltages are overlaid in FIG. 5B to exhibit different current signatures resulting from different target molecule-redox tag complexes, as provided herein. The redox tags of the four target molecules 510-1, 510-2, 510-3, and 510-4 may respond to different voltages, and thus the respective current signatures measured may be used to determine sequence 520 of the region of interest of the target substrate 220.

In some embodiments, the sensor may be configured to determine a residence time of the target molecule on the binding unit. In some cases, different target molecules or different tags coupled to the target molecules may exhibit different residence times to the binding unit of the sensor, and the residence time may be a unique or additional signature to analyze or identify the target molecules.

The sensor of the present disclosure may an electrical circuit (e.g., CMOS or FET circuit). The electrical circuit may be coupled to a voltage source. A constant voltage may be applied to the electrical circuit, and a change in the current may be measured. Alternatively, a change in voltage necessary to maintain a steady state current may be measured. The sensor may be in an electrolytic solution (e.g., 0.5 M Potassium Acetate and 10 mM KCl). Alternatively, the sensor may not be in an electrolytic solution. In some examples, the sensor may be in an aqueous solution or gas.

The one or more signals may be a current or voltage measured from the sensing circuit. The one or more signals may be a current and voltage measured from the sensing circuit. The signal may be a tunneling current. Alternatively, the signal may not be a tunneling current. The current may be a Faradaic current. Alternatively, the current may not be a Faradaic current. The current may be at least 1 picoamp (pA), 10 pA, 100 pA, 1 nanoamp (nA), 10 nA, 100 nA, 1 microamp (mA), 10 mA, 100 mA, or more. The current may be at most 100 mA, 10 mA, 1 mA, 100 nA, 10 nA, 1 nA, 100 pA, 10 pA, 1 pA, or less. The current may be at least in the picoamp (pA) range, tens of pA range, hundreds of pA range, nanoamp (nA) range, tens of nA range, hundreds of nA range, microamp (mA) range, tens of mA range, or higher. The current may be at most in the tens of mA range, mA range, hundreds of nA range, tens of nA range, nA range, hundreds of pA range, tens of pA range, pA range, or lower. The voltage may be at least 0.1 millivolt (mV), 0.5 mV, 1 mV, 5 mV, 10 mV, 50 mV, 100 mV, 500 mV, or more. The voltage may be at most 500 mV, 100 mV, 50 mV, 10 mV, 5 mV, 1 mV, 0.5 mV, 0.1 mV, or less. The voltage may be at least in the millivolt (mV) range, tens of mV range, hundreds of mV range, or higher. The voltage may be at most in the hundreds of mV range, tens of mV range, mV range, or lower.

In some embodiments, the sensor of the present disclosure may be provided as arrays, such as arrays present on a chip or biochip. The array of sensors may have any suitable number of any sensor of the present disclosure. The array may comprise about 10, about 20, about 50, about 100, about 200, about 400, about 600, about 800, about 1000, about 1500, about 2000, about 3000, about 4000, about 5000, about 10000, about 15000, about 20000, about 40000, about 60000, about 80000, about 100000, about 200000, about 400000, about 600000, about 800000, about 1000000, or more sensors.

In another aspect, the present disclosure provides a method for analyzing or identifying a target molecule. The method may comprise using a sensor to detect one or more signals indicative of an impedance or impedance change in the sensor when at least a portion of the target molecule is bound by at least a portion of the sensor. The method may further comprise using the one or more signals to analyze or identify the target molecule. The method may utilize any of the subject systems of the present disclosure, as illustrated in FIGS. 1 through 5.

In another aspect, the present disclosure provides a method for analyzing or identifying a target molecule. The method may comprise providing a sensor comprising a sensing electrode and a reference electrode in electrical communication with one another. The sensor may further comprise a dielectric material coupled to the sensing electrode and covering a first portion of a surface of the sensing electrode. The sensor may further comprise a conducting material coupled to the sensing electrode and covering a second portion of the surface of the sensing electrode. The sensor may further comprise a binding unit coupled to the conducting material, wherein the binding unit is configured to bind the target molecule. The method may further comprise detecting one or more signals indicative of an impedance or impedance change in the sensor when at least a portion of the target molecule is bound by the binding moiety. The method may further comprise using the one or more signals to analyze or identify the target molecule. The method may utilize any of the subject systems of the present disclosure, as illustrated in FIGS. 1 through 5.

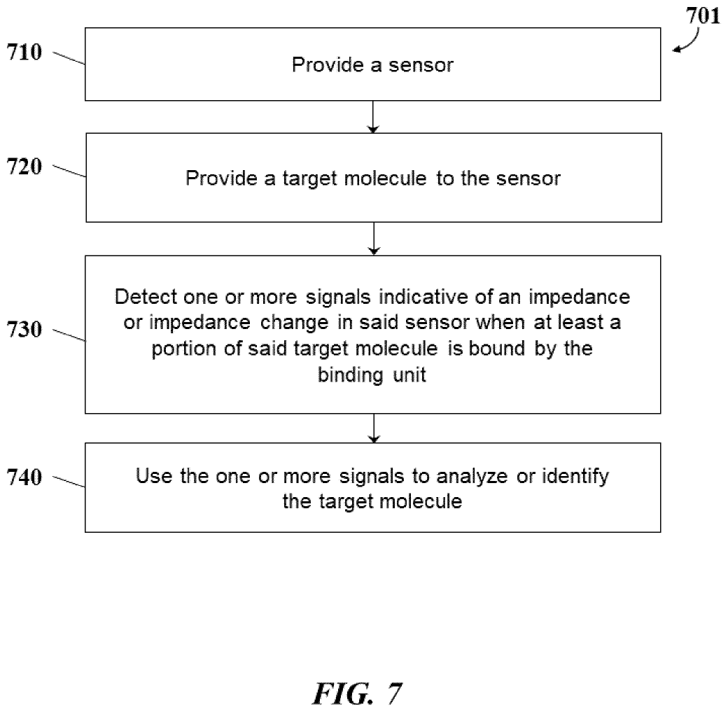

FIG. 7 shows an example process 701 of the method for analyzing or identifying a target molecule. The method may comprise providing a sensor (process 710). The sensor may comprise a binding unit. The method may comprise providing a target molecule to the sensor (process 720). The method may comprise detecting one or more signals indicative of an impedance or impedance change in the sensor when at least a portion of the target molecule is bound by the binding unit (process 730). The method may comprise using the one or more signals to analyze or identify the target molecule.

The one or more signals may be indicative of (i) electrical resistance or a change thereof in the sensor, (ii) electrical capacitance or a change thereof in the sensor, or (ii) an electrical inductance or a change thereof in the sensor. The one or more signals may be indicative of at least two of (i) electrical resistance or a change thereof in the sensor, (ii) electrical capacitance or a change thereof in the sensor, or (ii) an electrical inductance or a change thereof in the sensor. The one or more signals may be indicative of (i) electrical resistance or a change thereof in the sensor, (ii) electrical capacitance or a change thereof in the sensor, and (ii) an electrical inductance or a change thereof in the sensor.

The one or more signals may be current or voltage. The one or more signals may be current and voltage. The one or more signals may not be a tunneling current.

In some embodiments, the sensing electrode and the reference electrode may provide a first electric field. Additionally, the method may further comprise providing an additional electric field generator. The method may further comprise using the additional electric field generator to apply a second electric field in a second direction that is approximately perpendicular to a first direction of the first electric field.

In some embodiments, the method may further comprise determining a residence time of the target molecule on the binding unit. The method may further comprise using the residence time in place of and/or in addition to the one or more signals to analyze or identify the target molecule.

### II. Sample

Samples for analysis can comprise a plurality of polynucleotides. A polynucleotide can be single stranded DNA, double stranded DNA, or a combination thereof. The polynucleotides can comprise genomic DNA, genomic cDNA, cell free DNA, cell free cDNA, or a combination of any of the foregoing.

A polynucleotide can include cell-free DNA, circulating tumor DNA, genomic DNA, and DNA from formalin fixed and paraffin embedded (FFPE) samples. In some examples, an extracted DNA from a FFPE sample may be damaged, and such damaged DNA may be repaired by an available FFPE DNA repair kit. A sample can comprise any suitable DNA and/or cDNA sample such as for example, urine, stool, blood, saliva, tissue, biopsy, bodily fluid, or tumor cells.

The plurality of polynucleotides can be single-stranded or double-stranded.

A polynucleotide sample can be derived from any suitable source. For example, a sample can be obtained from a patient, from an animal, from a plant, or from the environment such as, for example, a naturally occurring or artificial atmosphere, a water system, soil, an atmospheric pathogen collection system, a sub-surface sediment, groundwater, or a sewage treatment plant.

Polynucleotides from a sample may include one more different polynucleotides, such as, for example, DNA, RNA, ribosomal RNA (rRNA), transfer RNA (tRNA), micro RNA (miRNA), messenger RNA (mRNA), fragments of any of foregoing, or combinations of any of the foregoing. A sample can comprise DNA. A sample can comprise genomic DNA. A sample can comprise mitochondrial DNA, chloroplast DNA, plasmid DNA, bacterial artificial chromosomes, yeast artificial chromosomes, oligonucleotide tags, or a combination of any of the foregoing.

The polynucleotides may be single-stranded, double-stranded, or a combination thereof. A polynucleotide can be a single-stranded polynucleotide, which may or may not be in the presence of double-stranded polynucleotides.

The starting amount of polynucleotides in a sample can be, for example, less than 50 ng, such as less than 45 ng, 40 ng, 35 ng, 30 ng, 25 ng, 20 ng, 15 ng, 10 ng, 5 ng, 4 ng, 3 ng, 2 ng, 1 ng, 0.5 ng, 0.1 ng, or less. The starting amount of polynucleotides in a sample can be, for example, more than 0.1 ng, such as more than 0.5 ng, 1 ng, 2 ng, 3 ng, 4 ng, 5 ng, 10 ng, 15 ng, 20 ng, 25 ng, 30 ng, 35 ng, 40 ng, 45 ng, 50 ng, or more. An amount of starting polynucleotides can be, for example, from 0.1 ng to 100 ng, from 1 ng to 75 ng, 5 ng to 50 ng, or from 10 ng to 20 ng.

The polynucleotides in a sample can be single-stranded, either as obtained or by way of treatment (e.g., denaturation). Further examples of suitable polynucleotides are described herein, such as with respect to any of the various aspects of the disclosure. Polynucleotides can be subjected to subsequent steps (e.g., circularization and amplification) without an extraction step, and/or without a purification step. For example, a fluid sample may be treated to remove cells without an extraction step to produce a purified liquid sample and a cell sample, followed by isolation of the polynucleotides from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, which may correspond to dead or damaged cells. The identity of such cells may be used to characterize the cells or population of cells from which they are derived, such as in a microbial community.

A sample can be from a subject. A subject can be any suitable organism including, for example, plants, animals, fungi, protists, monerans, viruses, mitochondria, and chloroplasts. Sample polynucleotides can be isolated from a subject, such as a cell sample, tissue sample, bodily fluid sample, or organ sample or cell cultures derived from any of these, including, for example, cultured cell lines, biopsy, blood sample, cheek swab, or fluid sample containing a cell such as saliva. The subject may be an animal such as a cow, a pig, a mouse, a rat, a chicken, a cat, a dog, or a mammal, such as a human. A sample can comprise tumor cells, such as in a sample of tumor tissue from a subject.

A sample may not comprise intact cells, can be treated to remove cells, or polynucleotides are isolated without a cellular extractions step such as to isolate cell-free polynucleotides, such as cell-free DNA.

Other examples of sample sources include those from blood, urine, feces, nares, the lungs, the gut, other bodily fluids or excretions, a derivative thereof, or a combination thereof.

A sample from a single individual can be divided into multiple separate samples, such as 2, 3, 4, 5, 6, 7, 8, 9, 10, or more separate samples that are subjected to methods of the disclosure independently, such as analysis in duplicate, triplicate, quadruplicate, or more. Where a sample is from a subject, a reference sequence may also be derived from the subject, such as a consensus sequence from the sample under analysis or the sequence of polynucleotides from another sample or tissue of the same subject. For example, a blood sample may be analyzed for ctDNA mutations, and cellular DNA from another sample from the subject such as a buccal or skin sample, can be analyzed to determine a reference sequence.

Polynucleotides can be extracted from a sample, with or without extraction from cells in a sample, according to any suitable method.

A plurality of polynucleotides can comprise cell-free polynucleotides, such as cell-free DNA (cfDNA) or circulating tumor DNA (ctDNA). Cell-free DNA circulates in both healthy and diseased individuals. cfDNA from tumors (ctDNA) is not confined to any specific cancer type, but appears to be a common finding across different malignancies. The free circulating DNA concentration in plasma can be lower in control subjects in comparison to that in patients having or suspected of having a condition. In an example, the free circulating DNA concentration in plasma can be, for example, from 14 ng/mL to 18 ng/mL in control subjects and from 18 ng/mL to 318 ng/mL in patients with neoplasia.

Apoptotic and necrotic cell death may contribute to cell-free circulating DNA in bodily fluids. For example, significantly increased circulating DNA levels may be observed in plasma of prostate cancer patients and other prostate diseases, such as Benign Prostate Hyperplasia and Prostatits. In addition, circulating tumor DNA may be present in fluids originating from the organs where the primary tumor occurs. In an example, breast cancer detection can be achieved in ductal lavages; colorectal cancer detection in stool; lung cancer detection in sputum, and prostate cancer detection in urine or ejaculate. Cell-free DNA may be obtained from a variety of sources. An example source may be blood samples of a subject. However, cfDNA or other fragmented DNA may be derived from a variety of other sources including, for example, urine and stool samples can be a source of cfDNA, including ctDNA.

In some embodiments, the target molecule may be indicative of a health condition or disease. In some cases, the disease may be tumor or cancer. Non-limiting examples of antigens which can be bound by the binding unit of the subject system may include, but are not limited to, 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-la), carbonic anhydrase 9 (CA-IX), Carcinoembryonic antigen (CEA), cardiac myosin, CCCTC-Binding Factor (CTCF), CCL 11 (eotaxin-1), CCR4, CCR5, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (a chain of IL-2receptor), CD27, CD274, CD28, CD3, CD3 P, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLECi2A), C-type lectin-like molecule-1 (CLL-1 or CLECL1), C—X—C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRL5), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-Rα), platelet-derived growth factor receptor β (PDGFR-β), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, R Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAPI, surviving, syndecan 1 (SDC1)+A314, SOX10, survivin, surviving-2B, synovial sarcoma, X breakpoint 2 (SSX2), T-cell receptor, TCR Γ Alternate Reading Frame Protein (TARP), telomerase, TEM1, tenascin C, TGF-β (e.g., TGF-β 1, TGF-β 2, TGF-β 3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, and κ-light chain.

### III. Nanopore

A sequencing system can include a reaction chamber that includes one or more nanopore devices. A nanopore device may be an individually addressable nanopore device. An individually addressable nanopore can be individually readable. An individually addressable nanopore can be individually writable. An individually addressable nanopore can be individually readable and individually writable. The system can include one or more computer processors for facilitating sample preparation and various operations of the disclosure, such as polynucleotide sequencing. The processor can be coupled to nanopore device.

A nanopore device may include a plurality of individually addressable sensing electrodes. Each sensing electrode can include a membrane adjacent to the electrode, and one or more nanopores in the membrane. A nanopore may be in a membrane such as a lipid bi-layer disposed adjacent or in sensing proximity to an electrode that is part of, or coupled to, an integrated circuit. A nanopore may be associated with an individual electrode and sensing integrated circuit or a plurality of electrodes and sensing integrated circuits. A nanopore can comprise a solid state nanopore.

Devices and systems for use in methods provided by the present disclosure may accurately detect individual nucleotide incorporation events, such as upon the incorporation of a nucleotide into a growing strand that is complementary to a template. An enzyme such as a DNA polymerase, RNA polymerase, or ligase can incorporate nucleotides to a growing polynucleotide chain. Enzymes such as polymerases can generate polynucleotide strands.

The added nucleotide can be complimentary to the corresponding template polynucleotide strand which is hybridized to the growing strand. A nucleotide can include a tag or tag species that is coupled to any location of the nucleotide including, but not limited to a phosphate such as a γ-phosphate, sugar or nitrogenous base moiety of the nucleotide. In some cases, tags are detected while tags are associated with a polymerase during the incorporation of nucleotide tags. The tag may continue to be detected until the tag translocates through the nanopore after nucleotide incorporation and subsequent cleavage and/or release of the tag. Nucleotide incorporation events can release tags from the nucleotides which pass through a nanopore and are detected. A tag can be released by the polymerase, or cleaved/released in any suitable manner including without limitation cleavage by an enzyme located near the polymerase. In this way, the incorporated base may be identified (i.e., A, C, G, T or U) because a unique tag is released from each type of nucleotide (i.e., adenine, cytosine, guanine, thymine or uracil). In nucleotide incorporation events that do not release, a tag coupled to an incorporated nucleotide is detected with the aid of a nanopore. In some examples, the tag can move through or in proximity to the nanopore and be detected with the aid of the nanopore.

Methods and systems of the disclosure can enable the detection of polynucleotide incorporation events, such as at a resolution of at least 1, 2, 3, 4, 5, 6, 7, 8, 9, 10, 20, 30, 40, 50, 100, 500, 1000, 5000, 10000, 50000, or 100000 polynucleotide bases within a given time period. For example, a nanopore device can be used to detect individual polynucleotide incorporation events, with each event being associated with an individual nucleic acid base. In other examples, a nanopore device can be used to detect an event that is associated with a plurality of bases. For example, a signal sensed by the nanopore device can be a combined signal from at least 2, 3, 4, or 5 bases.

In certain sequencing methods, tags do not pass through the nanopore. The tags can be detected by the nanopore and exit the nanopore without passing through the nanopore such as exiting from the inverse direction from which the tag entered the nanopore. A sequencing device can be configured to actively expel the tags from the nanopore.

In certain sequencing methods tags are not released upon nucleotide incorporation events. Nucleotide incorporation events can present tags to a nanopore without releasing the tags. The tags can be detected by the nanopore without being released. The tags may be attached to the nucleotides by a linker of sufficient length to present the tag to the nanopore for detection.

Nucleotide incorporation events may be detected in real-time as they occur by a nanopore. An enzyme such as a DNA polymerase attached to or in proximity to a nanopore can facilitate the flow of a polynucleotide through or adjacent to a nanopore. A nucleotide incorporation event, or the incorporation of a plurality of nucleotides, may release or present one or more tags, which may be detected by a nanopore. Detection can occur as the tags flow through or adjacent to the nanopore, as the tags reside in the nanopore and/or as the tags are presented to the nanopore. In some cases, an enzyme attached to or in proximity to the nanopore may aid in detecting tags upon the incorporation of one or more nucleotides.

A tag can be an atom, a molecule, a collection of atoms, or a collection of molecules. A tag may provide an optical, electrochemical, magnetic, or electrostatic such as an inductive or capacitive, signature, which signature may be detected with the aid of a nanopore.

The nanopore may be formed or otherwise embedded in a membrane disposed adjacent to a sensing electrode of a sensing circuit, such as an integrated circuit. An integrated circuit may be an application specific integrated circuit (ASIC). An integrated circuit can be a field effect transistor or a complementary metal-oxide semiconductor (CMOS). A sensing circuit may be situated in a chip or other device having the nanopore, or off of the chip or device, such as in an off-chip configuration.

As a nucleic acid or tag flows through or adjacent to the nanopore, the sensing circuit detects an electrical signal associated with the nucleic acid or tag. The nucleic acid may be a subunit of a larger strand. The tag may be a byproduct of a nucleotide incorporation event or other interaction between a tagged nucleic acid and the nanopore or a species adjacent to the nanopore, such as an enzyme that cleaves a tag from a nucleic acid. The tag may remain attached to the nucleotide. A detected signal may be collected and stored in a memory location, and later used to construct a sequence of the nucleic acid. The collected signal may be processed to account for any abnormalities in the detected signal, such as errors.

Nanopores may be used to sequence polynucleotides indirectly, in some cases with electrical detection. Indirect sequencing may be any method where an incorporated nucleotide in a growing strand does not pass through the nanopore. The polynucleotide may pass within any suitable distance from and/or proximity to the nanopore, in some cases within a distance such that tags released from nucleotide incorporation events are detected in the nanopore.

Byproducts of nucleotide incorporation events may be detected by the nanopore. Nucleotide incorporation events refer to the incorporation of a nucleotide into a growing polynucleotide chain. A byproduct may be correlated with the incorporation of a given type nucleotide. Nucleotide incorporation events can be catalyzed by an enzyme, such as DNA polymerase, and use base pair interactions with a template molecule to choose amongst the available nucleotides for incorporation at each location.

A nucleic acid sample may be sequenced using tagged nucleotides or nucleotide analogs. In some examples, a method for sequencing a nucleic acid molecule comprises (a) incorporating (e.g., polymerizing) tagged nucleotides, wherein a tag associated with an individual nucleotide is released upon incorporation, and (b) detecting the released tag with the aid of a nanopore. In some instances, the method further comprises directing the tag attached to or released from an individual nucleotide through the nanopore. The released or attached tag may be directed by any suitable technique, in some cases with the aid of an enzyme (or molecular motor) and/or a voltage difference across the pore. Alternative, the released or attached tag may be directed through the nanopore without the use of an enzyme. For example, the tag may be directed by a voltage difference across the nanopore as described herein.

A tag may be detected with the aid of a nanopore device having at least one nanopore in a membrane. The tag may be associated with an individual tagged nucleotide during incorporation of the individual tagged nucleotide. A nanopore device can detect a tag associated with an individual tagged nucleotide during incorporation. The tagged nucleotides, whether incorporated into a growing nucleic acid strand or unincorporated, can be detected, determined, or differentiated for a given period of time by the nanopore device, in some cases with the aid of an electrode and/or nanopore of the nanopore device. The time period within which the nanopore device detects the tag may be shorter, in some cases substantially shorter, than the time period in which the tag and/or nucleotide coupled to the tag is held by an enzyme, such as an enzyme facilitating the incorporation of the nucleotide into a nucleic acid strand (e.g., a polymerase). A tag can be detected by the electrode a plurality of times within the time period that the incorporated tagged nucleotide is associated with the enzyme. For instance, the tag can be detected by the electrode at least 1, 2, 3, 4, 5, 6, 7, 8, 9, 10, 20, 30, 40, 50, 60, 70, 80, 90, 100, 200, 300, 400, 500, 1000, 10,000, 100,000, or 1,000,000 times within the time period that the incorporated tagged nucleotide is associated with the enzyme.

Sequencing can be accomplished using pre-loaded tags. Pre-loading a tag can comprise directing at least a portion of the tag through at least a portion of a nanopore while the tag can be attached to a nucleotide, which nucleotide has been incorporated into a nucleic acid strand (e.g., growing nucleic acid strand), is undergoing incorporation into the nucleic acid strand, or has not yet been incorporated into the nucleic acid strand but may undergo incorporation into the nucleic acid strand. Pre-loading a tag can comprise directing at least a portion of the tag through at least a portion of the nanopore before the nucleotide has been incorporated into the nucleic acid strand or while the nucleotide is being incorporated into the nucleic acid strand. Pre-loading a tag can include directing at least a portion of the tag through at least a portion of the nanopore after the nucleotide has been incorporated into the nucleic acid strand.

A tag associated with an individual nucleotide can be detected by a nanopore without being released from the nucleotide upon incorporation. Tags can be detected without being released from incorporated nucleotides during synthesis of a nucleic acid strand that is complementary to a target strand. The tags can be attached to the nucleotides with a linker such that the tag is presented to the nanopore (e.g., the tag hangs down into or otherwise extend through at least a portion of the nanopore). The length of the linker may be sufficiently long so as to permit the tag to extend to or through at least a portion of the nanopore. In some instances, the tag is presented to (i.e., moved into) the nanopore by a voltage difference. Other ways to present the tag into the pore may also be suitable (e.g., use of enzymes, magnets, electric fields, pressure differential). In some instances, no active force is applied to the tag (i.e., the tag diffuses into the nanopore).

A chip for sequencing a nucleic acid sample can comprise a plurality of individually addressable nanopores. An individually addressable nanopore of the plurality can contain at least one nanopore formed in a membrane disposed adjacent to an integrated circuit. Each individually addressable nanopore can be capable of detecting a tag associated with an individual nucleotide. The nucleotide can be incorporated (e.g., polymerized) and the tag may not be released from the nucleotide upon incorporation.

Tags can be presented to the nanopore upon nucleotide incorporation events and are released from the nucleotide. The released tags can go through the nanopore. The tags do not pass through the nanopore in some instances. A tag that has been released upon a nucleotide incorporation event is distinguished from a tag that may flow through the nanopore, but has not been released upon a nucleotide incorporation event at least in part by the dwell time in the nanopore. In some cases, tags that dwell in the nanopore for at least 100 milliseconds (ms) are released upon nucleotide incorporation events and tags that dwell in the nanopore for less than 100 ms are not released upon nucleotide incorporation events. Tags may be captured and/or guided through the nanopore by a second enzyme or protein (e.g., a nucleic acid binding protein). The second enzyme may cleave a tag upon (e.g., during or after) nucleotide incorporation. A linker between the tag and the nucleotide may be cleaved.

A tag that is coupled to an incorporated nucleotide is distinguished from a tag associated with a nucleotide that has not been incorporated into a growing complementary strand based on the residence time of the tag in the nanopore or a signal detected from the unincorporated nucleotide with the aid of the nanopore. An unincorporated nucleotide may generate a signal (e.g., voltage difference, current) that is detectable for a time period between 1 nanosecond (ns) and 100 ms, or between 1 ns and 50 ms, whereas an incorporated nucleotide may generate a signal with a lifetime between 50 ms and 500 ms, or 100 ms and 200 ms. An unincorporated nucleotide may generate a signal that is detectable for a time period between 1 ns and 10 ms, or 1 ns and 1 ms. An unincorporated tag is detectable by a nanopore for a time period (average) that is longer than the time period in which an incorporated tag is detectable by the nanopore.

Incorporated nucleic acids can be detected by and/or are detectable by the nanopore for a shorter period of time than an un-incorporated nucleotide. Alternatively, incorporated nucleic acids can be detected by and/or are detectable by the nanopore for a longer period of time than an un-incorporated nucleotide. The difference and/or ratio between these times can be used to determine whether a nucleotide detected by the nanopore is incorporated or not, as described herein.

The detection period can be based on the free-flow of the nucleotide through the nanopore; an unincorporated nucleotide may dwell at or in proximity to the nanopore for a time period between 1 nanosecond (ns) and 100 ms, or between 1 ns and 50 ms, whereas an incorporated nucleotide may dwell at or in proximity to the nanopore for a time between 50 ms and 500 ms, or 100 ms and 200 ms. The time periods can vary based on processing conditions; however, an incorporated nucleotide may have a dwell time that is greater than that of an unincorporated nucleotide.

A tag or tag species can include a detectable atom or molecule, or a plurality of detectable atoms or molecules. A tag can include a one or more adenine, guanine, cytosine, thymine, uracil, or a derivative thereof linked to any position including a phosphate group, sugar or a nitrogenous base of a nucleic acid molecule. A tag can include one or more adenine, guanine, cytosine, thymine, uracil, or a derivative thereof covalently linked to a phosphate group of a nucleic acid base.

A tag can have a length of at least 0.1 nanometers (nm), 1 nm, 2 nm, 3 nm, 4, nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 200 nm, 300 nm, 400 nm, 500 nm, or 1000 nm.

A tag can include a tail of repeating subunits, such as a plurality of adenine, guanine, cytosine, thymine, uracil, or a derivative thereof. For example, a tag can include a tail portion having at least 2, 3, 4, 5, 6, 7, 8, 9, 10, 20, 30, 40, 50, 60, 70, 80, 90, 100, 1000, 10,000, or 100,000 subunits of adenine, guanine, cytosine, thymine, uracil, or a derivative thereof. The subunits can be linked to one another, and at a terminal end linked to a phosphate group of the nucleic acid. Other examples of tag portions include any polymeric material, such as polyethylene glycol (PEG), polysulfonates, amino acids, or any completely or partially positively charged, negatively charged, or un-charged polymer.

### IV. Polymerase

A DNA polymerase can be bound to the 3′ end of a nicked strand of the polynucleotide at the nicking site. DNA sequencing can be accomplished by using an enzyme such as a DNA polymerize to amplify and transcribe a polynucleotide in proximity to a nanopore and tagged nucleotides. Sequencing methods can involve incorporating or polymerizing tagged nucleotides using a polymerase such as a DNA polymerase, or transcriptase. The polymerase can be mutated to allow it to accept tagged nucleotides. The polymerase can also be mutated to increase the time for which the tag is detected by the nanopore.

A sequencing enzyme can be, for example, any suitable enzyme that creates a polynucleotide strand by phosphate linkage of nucleotides. The DNA polymerase can be, for example, a 9° Nm™ polymerase or a variant thereof, an E. Coli DNA polymerase I, a Bacteriophage T4 DNA polymerase, a Sequenase, a Taq DNA polymerase, a 9° Nm™ polymerase (exo-)A485L/Y409V, a Φ29 DNA Polymerase, a Bst DNA polymerase, or variants, mutants, or homologs of any of the foregoing. A homolog can have any suitable percentage homology such as, for example, at least 30%, at least 40%, at least 50%, at least 60%, at least 70%, at least 80%, at least 90%, or at least 95% sequence identity.

In some examples, for nanopore sequencing, a polymerization enzyme can be attached to or situated in proximity to a nanopore. Suitable methods for attaching the polymerization enzyme to a nanopore include cross-linking the enzyme to the nanopore or in proximity to the nanopore such as via the formation of intra-molecular disulfide bonds. The nanopore and the enzyme may also be a fusion such as an encoded by a single polypeptide chain. Methods for producing fusion proteins may include fusing the coding sequence for the enzyme in frame and adjacent to the coding sequence for the nanopore and expressing this fusion sequence from a single promoter. A polymerization enzyme can be attached or coupled to a nanopore using molecular staples or protein fingers. A polymerization enzyme can be attached to a nanopore via an intermediate molecule, such as for example biotin conjugated to both the enzyme and the nanopore with streptavidin tetramers linked to both biotins. The intermediate molecule can be referred to as a linker.

The sequencing enzyme can also be attached to a nanopore with an antibody. Proteins that form a covalent bond between each other can be used to attach a polymerase to a nanopore. Phosphatase enzymes or an enzyme that cleaves a tag from a nucleotide can also be attached to the nanopore.

The polymerase can be mutated to facilitate and/or to improve the efficiency of the mutated polymerase for incorporation of tagged nucleotides into a growing polynucleotide relative to the non-mutated polymerase. The polymerase can be mutated to improve entry of the nucleotide analog such as a tagged nucleotide, into the active site region of the polymerase and/or mutated for coordinating with the nucleotide analogs in the active region.

Other mutations such as amino acid substitutions, insertions, deletions, and/or exogenous features to a polymerize can result in enhanced metal ion coordination, reduced exonuclease activity, reduced reaction rates at one or more steps of the polymerase kinetic cycle, decreased branching fraction, altered cofactor selectivity, increased yield, increased thermostability, increased accuracy, increased speed, increased read length, increased salt tolerance relative to the non-mutated polymerase.

A suitable polymerase can have a kinetic rate profile that is suitable for detection of the tags by a nanopore. The rate profile generally refers to the overall rate of nucleotide incorporation and/or a rate of any step of nucleotide incorporation such as nucleotide addition, enzymatic isomerization such as to or from a closed state, cofactor binding or release, product release, incorporation of polynucleotide into the growing polynucleotide, or translocation.

A polymerase can be adapted to permit the detection of sequencing events. The rate profile of a polymerase can be such that a tag is loaded into (and/or detected by) the nanopore for an average of 0.1 milliseconds (ms), 1 ms, 5 ms 10 ms, 20 ms, 30 ms, 40 ms, 50 ms, 60 ms, 80 ms, 100 ms, 120 ms, 140 ms, 160 ms, 180 ms, 200 ms, 220 ms, 240 ms, 260 ms, 280 ms, 300 ms, 400 ms, 500 ms, 600 ms, 800 ms, or 1000 ms. For example, the rate profile of a polymerase can be such that a tag is loaded into and/or detected by the nanopore for an average of at least 5 ms, at least 10 ms, at least 20 ms, at least 30 ms, at least 40 ms, at least 50 ms, at least 60 ms, at least 80 ms, at least 100 ms, at least 120 ms, at least 140 ms, at least 160 ms, at least 180 ms, at least 200 ms, at least 220 ms, at least 240 ms, at least 260 ms, at least 280 ms, at least 300 ms, at least 400 ms, at least 500 ms, at least 600 ms, at least 800 ms, or at least 1000 ms. A tag can be detected by the nanopore for an average between 80 ms and 260 ms, between 100 ms and 200 ms, or between 100 ms and 150 ms.

A nanopore/polymerase complex can be configured to permit the detection of one or more events associated with amplification and transcription of the circular polynucleotide. The one or more events may be kinetically observable and/or non-kinetically observable such as a nucleotide migrating through a nanopore without coming in contact with a polymerase.

In some cases, the polymerase reaction exhibits two kinetic steps which proceed from an intermediate in which a nucleotide or a polyphosphate product is bound to the polymerase enzyme, and two kinetic steps which proceed from an intermediate in which the nucleotide and the polyphosphate product are not bound to the polymerase enzyme. The two kinetic steps can include enzyme isomerization, nucleotide incorporation, and product release. In some cases, the two kinetic steps are template translocation and nucleotide binding.

A suitable polymerase can exhibit strong or enhanced strand displacement.

### V. Identification of Sequence Variants

Methods provided by the present disclosure can be used to identify sequence variants in a polynucleotide sample. A sequence difference between sequencing reads and a reference sequence is referred to as a genuine sequence variant if the sequence difference occurs in at least two different polynucleotides, e.g., two different circular polynucleotides, which can be distinguished as a result of having different junctions. Because the position and type of a sequence variant that are the result of amplification or sequencing errors are unlikely to be duplicated exactly on two different polynucleotides comprising the same target sequence, including this validation parameter can reduce the background of erroneous sequence variants, with a concurrent increase in the sensitivity and accuracy of detecting actual sequence variation in a sample. A sequence variant can have a frequency less than 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower is sufficiently above background to permit an accurate identification. A sequence variant can occur with a frequency of less than 0.1%. The frequency of a sequence variant can be sufficiently above background when such frequency is statistically significantly above the background error rate, for example, with a p-value less than 0.05, 0.01, 0.001, or 0.0001. The frequency of a sequence variant can be sufficiently above background when the frequency is at least 2-fold, 3-fold, 4-fold, 5-fold, 6-fold, 7-fold, 8-fold, 9-fold, 10-fold, 25-fold, 50-fold, 100-fold, or more above the background error rate. The background error rate for accurately determining the sequence at a given position can be less than 1%, 0.5%, 0.1%, 0.05%, 0.01%, 0.005%, 0.001%, or 0.0005%.

Identifying a sequence variant can comprise optimally aligning one or more sequencing reads with a reference sequence to identify differences between the two, as well as to identify junctions. Alignment can involve placing one sequence along another sequence, iteratively introducing gaps along each sequence, scoring how well the two sequences match, and repeating for various positions along the reference. The best-scoring match is deemed to be the alignment and represents an inference about the degree of relationship between the sequences.

A reference sequence to which sequencing reads are compared is a reference genome, such as the genome of a member of the same species as the subject. A reference genome may be complete or incomplete. A reference genome can consist only of regions containing target polynucleotides, such as from a reference genome or from a consensus generated from sequencing reads under analysis. A reference sequence can comprise or can consist of sequences of polynucleotides of one or more organisms, such as sequences from one or more bacteria, archaea, viruses, protists, fungi, or other organism. A reference sequence can consist of only a portion of a reference genome, such as regions corresponding to one or more target sequences under analysis. For example, for detection of a pathogen, a reference genome can be the entire genome of the pathogen, or a portion thereof useful in identification, such as of a particular strain or serotype. A sequencing read can be aligned to multiple different reference sequences, such as to screen for multiple different organisms or strains.

### VI. Therapeutic Applications

Methods, systems, and compositions provided herein can be directed to one or more therapeutic applications, such as in the characterization of a patient sample and optionally diagnosis of a condition of a subject. Therapeutic applications can include informing the selection of therapies to which a patient may be most responsive and/or treatment of a subject in need of therapeutic intervention based on the results of methods provided by the present disclosure.

For example, methods provided by the present disclosure can be used to diagnose tumor presence, progression and/or metastasis of tumors, such as when the polynucleotides analyzed comprise or consist of cfDNA, ctDNA, or fragmented tumor DNA. A subject may be monitored for tumor treatment efficacy, for example, by monitoring ctDNA over time, a decrease in ctDNA can be used as an indication of treatment efficacy, and increases in ctDNA can inform selection of different treatments and/or different dosages. Other uses include evaluations of organ rejection in transplant recipients such as where increases in the amount of circulating DNA corresponding to the transplant donor genome is used as an early indicator of transplant rejection, and genotyping/isotyping of pathogen infections, such as viral or bacterial infections. Detection of sequence variants in circulating fetal DNA may be used to diagnose a condition of a fetus.

Methods provided by the present disclosure can comprise diagnosing a subject based on a result of the sequencing, such as diagnosing the subject with a disease associated with a detected causal genetic variant, or reporting a likelihood that the patient has or will develop such disease.

A causal genetic variant can include sequence variants associated with a particular type or stage of cancer, or of cancer having a particular characteristic such as metastatic potential, drug resistance, and/or drug responsiveness. Methods provided by the present disclosure can be used to inform therapeutic decisions, guidance and monitoring, of cancer therapies. For example, treatment efficacy can be monitored by comparing patient ctDNA samples from before, during, and after treatment with particular including molecular targeted therapies such as monoclonal drugs, chemotherapeutic drugs, radiation protocols, and combinations of any of the foregoing. For example, the ctDNA can be monitored to see if certain mutations increase or decrease, or new mutations appear, after treatment, which can allow a physician to modify a treatment in a much shorter period of time than afforded by methods of monitoring that track patient symptoms. Methods can comprise diagnosing a subject based on the results of polynucleotide sequencing, such as diagnosing the subject with a particular stage or type of cancer associated with a detected sequence variant, or reporting a likelihood that the patient has or will develop such cancer.

For example, for therapies that are specifically targeted to patients on the basis of molecular markers, patients can be tested to find out if certain mutations are present in their tumor, and these mutations can be used to predict response or resistance to the therapy and guide the decision whether to use the therapy. Detecting and monitoring ctDNA during the course of treatment can be useful in guiding treatment selections.

Sequence variants associated with one or more kinds of cancer that may be used for diagnosis, prognosis, or treatment decisions. For example, suitable target sequences of oncological significance include alterations in the TP53 gene, the ALK gene, the KRAS gene, the PIK3CA gene, the BRAF gene, the EGFR gene, and the KIT gene. A target sequence the may be specifically amplified, and/or specifically analyzed for sequence variants may be all or part of a cancer-associated gene.

Methods provided by the present disclosure can be useful in discovering new, rare mutations that are associated with one or more cancer types, stages, or cancer characteristics. For example, in populations of individuals sharing a characteristic under analysis such as a particular disease, type of cancer, and/or stage of cancer, using methods provided by the present disclosure sequence variants can be identified reflecting mutations in particular genes or parts of genes. Identified sequence variants occurring with a statistically significantly greater frequency among the group of individuals sharing the characteristic than in individuals without the characteristic may be assigned a degree of association with that characteristic. The sequence variants or types of sequence variants so identified may then be used in diagnosing or treating individuals discovered to harbor them.

Additional therapeutic applications can include use in non-invasive fetal diagnostics. Fetal DNA can be found in the blood of a pregnant woman. Methods provided by the present disclosure can be used to identify sequence variants in circulating fetal DNA, and thus may be used to diagnose one or more genetic diseases in the fetus, such as those associated with one or more causal genetic variants. Examples of causal genetic variants include trisomies, cystic fibrosis, sickle-cell anemia, and Tay-Saks disease. The mother may provide a control sample and a blood sample to be used for comparison. The control sample may be any suitable tissue, and can then be sequenced to provide a reference sequence. Sequences of cfDNA corresponding to fetal genomic DNA can then be identified as sequence variants relative to the maternal reference. The father may also provide a reference sample to aid in identifying fetal sequences, and sequence variants.

Different therapeutic applications can include detection of exogenous polynucleotides, including from pathogens such as bacteria, viruses, fungi, and microbes, which information may inform a treatment.

### VII. Computer Systems

The present disclosure provides computer systems that are programmed to implement one or more methods of the present disclosure. Computer systems of the present disclosure may be used to regulate various operations of the sensor, such as detecting one or more signals indicative of an impedance or impedance change in the sensor when at least a portion of a target molecule is bound by a binding moiety of the sensor.

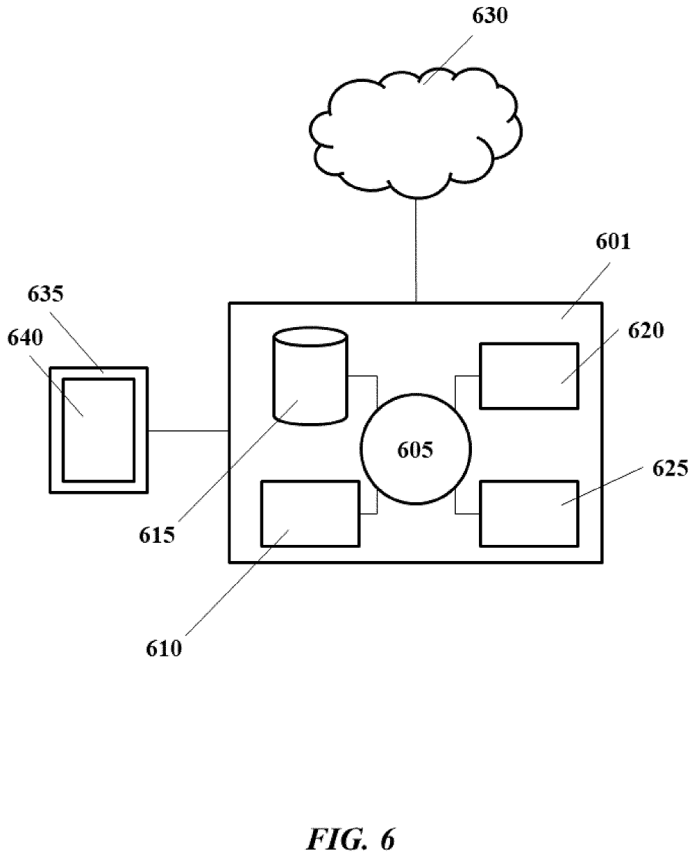

FIG. 6 shows a computer system 601 that is programmed or otherwise configured to communicate with and regulate various aspects of sequencing of the present disclosure. The computer system 601 can communicate with, for example, one or more circuitry coupled to or comprising the sensor, and one or more devices (e.g., machines) used to prepare, treat, or keep one or more reaction mixtures for the sensing. The computer system 601 may also communicate with one or more controllers or processors of the present disclosure. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, (i) progress of the reaction mixture, (ii) progress of sequencing, and (iii) sequencing information obtained from sequencing. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, determine sequence readout of a target nucleotide, polynucleotide, peptide, polypeptide, protein, etc.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-32. (canceled)

33. A method for analyzing a nucleic acid molecule, comprising:
(a) providing a sensor comprising (i) a membrane comprising a nanopore and (ii) an electrode in proximity to said membrane, wherein said nucleic acid molecule is disposed in proximity to said nanopore;
(b) using a polymerizing enzyme to perform an extension reaction on said nucleic acid molecule disposed in proximity to said nanopore, wherein said extension reaction comprises incorporating a nucleotide comprising a tag into a growing strand that is complementary to at least a portion of said nucleic acid molecule;
(c) using said electrode and said nanopore to measure an impedance value or change thereof during incorporation of said nucleotide into said growing strand; and
(d) using said impedance value or change thereof measured in (c) to identify said nucleotide, thereby identifying at least a portion of a sequence of said nucleic acid molecule.

34. The method of claim 33, wherein said membrane is a lipid membrane.

35. The method of claim 33, wherein membrane is a polymeric membrane.

36. The method of claim 33, wherein said polymerizing enzyme is coupled to said nanopore.

37. The method of claim 33, wherein identification of said nucleotide in (d) is based on a plurality of measurements of said impendence value or change thereof during incorporation of said nucleotide into said growing strand.

38. The method of claim 33, wherein the plurality of measurements is measured within a time period ranging between about 100 microseconds and about 10 milliseconds.

39. The method of claim 33, further comprising identifying said nucleotide based on a residence time of said nucleotide in proximity to said polymerizing enzyme, to identify said at least said portion of said sequence of said nucleic acid molecule.

40. The method of claim 33, further comprising a reference electrode in electrical communication with electrode, wherein (i) said electrode and said reference electrode are disposed on opposite sides of said membrane, and (ii) said impedance value or change thereof is measured between said electrode and said reference electrode.

41. The method of claim 33, wherein said nanopore is a protein nanopore.

42. The method of claim 33, wherein said impedance value or change thereof is at least about 1,000 kiloohms.

43. A system for analyzing a nucleic acid molecule, comprising:
a sensor comprising:
(i) a membrane comprising a nanopore;
(ii) a polymerizing enzyme configured to perform an extension reaction on said nucleic acid molecule when said nucleic acid molecule is disposed in proximity to said nanopore, wherein said extension reaction comprises incorporating a nucleotide comprising a tag into a growing strand that is complementary to at least a portion of said nucleic acid molecule; and
(iii) an electrode in proximity to said nanopore; and
a processor operatively coupled to said sensor, wherein said processor is configured (1) to direct said electrode and said nanopore to measure an impedance value or change thereof during incorporation of said nucleotide into said growing strand, and (2) use said impedance value or change thereof that is measured in (1) to identify said nucleotide, thereby identifying at least a portion of a sequence of said nucleic acid molecule.

44. The system of claim 43, wherein said membrane is a lipid membrane.

45. The system of claim 43, wherein membrane is a polymeric membrane.

46. The system of claim 43, wherein said polymerizing enzyme is coupled to said nanopore.

47. The system of claim 43, wherein said processor is configured to identify said nucleotide based on a plurality of measurements of said impendence value or change thereof during incorporation of said nucleotide into said growing strand.

48. The system of claim 47, wherein the plurality of measurements is measured within a time period ranging between about 100 microseconds and about 10 milliseconds.

49. The system of claim 43, wherein said processor is configured to identify said nucleotide based on a residence time of said nucleotide in proximity to said polymerizing enzyme, to identify said at least said portion of said sequence of said nucleic acid molecule.

50. The system of claim 43, further comprising a reference electrode in electrical communication with electrode, wherein (i) said electrode and said reference electrode are disposed on opposite sides of said membrane, and (ii) said impedance value or change thereof is measured between said electrode and said reference electrode.

51. The system of claim 43, wherein said nanopore is a protein nanopore.

52. The system of claim 43, wherein said impedance value or change thereof is at least about 1,000 kiloohms.
